(19) United States
(12) Patent Application Publication
(10) Pub. No.: US 2024/0290442 A1
(43) Pub. Date: Aug. 29, 2024

(54) Selecting Clinical Trial Sites Based on Multiple Target Variables Using Machine Learning

(71) Applicant: Janssen Research & Development, LLC, Raritan, NJ (US)

(72) Inventors: Kaitlin Ann Hood, Philadelphia, PA (US); Francisco Xavier Talamas, Livingston, NJ (US); Geoffrey Jerome Kip, Philadelphia, PA (US); Hans Roeland Geert Wim Verstraete, Shilde (BE)

(21) Appl. No.: 18/689,855

(22) PCT Filed: Sep. 9, 2022
(86) PCT No.: PCT/IB2022/058525

§ 371 Date: Mar. 6, 2024

Related U.S. Application Data
(60) Provisional application No. 63/242,753, filed on Sep. 10, 2021.

(51) Int. Cl. G16H 10/20; G06N 20/00

(52) U.S. Cl. CPC G16H 10/20; G06N 20/00

(57) ABSTRACT

Disclosed herein are methods for generating an automated method for determining or selecting one or more clinical trial sites for inclusion in a clinical trial. The method includes generating a predicted site enrollment (e.g., number of patients a site will enroll) and a predicted site default likelihood (e.g., how likely a site is to enroll zero patients or fewer patients than a predetermined threshold) for clinical trial sites by applying one or more machine learning models. The method further includes ranking the one or more clinical trial sites according to the predicted site enrollment and the predicted site default likelihood for the one or more clinical trial sites; and selecting top-ranked clinical trial sites.

### CROSS-REFERENCE TO RELATED APPLICATIONS

This application claims the benefit of U.S. Provisional Application No. 63/242,753 filed on Sep. 10, 2021, which is incorporated by reference herein.

### BACKGROUND

Performing site selection for clinical trials is a valuable step for ensuring on-time and on-target enrollment completion. Sluggish patient recruitment may disrupt clinical trial timelines and affect a clinical trial site's performance. Relying solely on historical performance has shown to be a weak predictor of a site's future performance and of a trial's overall timeline. To deliver robust predictions of a site's enrollment, an advanced analytics platform to assist site selection and planning is needed.

### SUMMARY

As described herein, systems, non-transitory computer readable media, and methods are used to predict target variables informative of site enrollment (e.g., number of patients a site will enroll) and site default likelihood (e.g., how likely a site is to enroll zero patients or fewer patients than a predetermined threshold) of one or more clinical trial sites. The prediction(s) can be used to assist selection of trial sites (e.g., healthcare facility and principal investigator pairs) for planning and supporting one or more clinical trials. The systems and methods described herein involve the engineering of features, predicting the two target variables that include any of enrolled patients, enrollment rate, default, and/or site agility using machine learning models, and ranking of sites that improve the selection of clinical trial sites that are likely to be successful.

Various embodiments disclosed herein involve building machine learning models including features that are selected through a specific feature selection process. Namely, the feature selection process involves generating features of historical clinical trial data over time periods in relation to reference time windows and reference entities, and selecting top features for inclusion in machine learning models. In various embodiments, the systems and methods select top-performing models from among a large selection of machine learning models. Predictions from the top-performing models are visualized, e.g., in quadrant graphs to elucidate site rankings. Simulations of patient enrollment capture the stochastic fluctuations in multi-site enrollment timelines with limited assumptions, producing statistically-robust enrollment curves. The final output may be a ranked list of sites with corresponding contact information to deliver to feasibility stakeholders. This final output assists in identifying and prioritizing the best performing sites for enrollment of patients for a specific clinical trial.

Disclosed herein is an automated method for determining or selecting one or more clinical trial sites for inclusion in a clinical trial, comprising: obtaining input data comprising data of an upcoming trial protocol, for each of the one or more clinical trial sites: generating a predicted site enrollment and a predicted site default likelihood for the clinical trial site by applying one or more machine learning models to selected features of the input data: ranking the one or more clinical trial sites according to the predicted site enrollment and the predicted site default likelihood for the one or more clinical trial sites; and selecting top-ranked clinical trial sites, wherein each of the selected clinical trial sites has a predicted site enrollment above a first threshold value and a predicted site default likelihood below a second threshold value, wherein the selected features are previously determined by performing feature engineering on historical clinical trial data.

In various embodiments, the first threshold value is a median predicted site enrollment across the one or more clinical trial sites or a first specified value, and wherein the second threshold value is a median predicted site default likelihood across the one or more clinical trial sites or a second specified value.

In various embodiments, the method further comprising visualizing the predicted site enrollment and the predicted site default likelihood for the clinical trial sites in a quadrant graph.

In various embodiments, the method further comprising generating a plurality of quantitative values informative of predicted enrollment timelines by applying a stochastic model to the predicted site enrollment and the predicted site default likelihood for the one or more clinical trial sites.

In various embodiments, the simulation method comprises Monte Carlo simulation.

In various embodiments, the plurality of quantitative values informative of predicted enrollment timeline comprises one or more of time to enroll between 50-1000 patients, number of patients enrolled in 4 months, number of patients enrolled in 12 months, number of patients enrolled in 18 months, number of patients enrolled in 24 months, and number of patients enrolled between 3-48 months.

In various embodiments, the predicted site enrollment and the predicted site default likelihood are validated by using one or more of the historical clinical trial data and prospective clinical trial data.

In various embodiments, the method further comprises an improvement of at least 11% in identifying the top-ranked clinical trial sites.

In various embodiments, the method further comprises generating a site list of the selected top-ranked clinical trial sites.

In various embodiments, the site list comprises corresponding contact information useful for feasibility stakeholders.

In various embodiments, the one or more machine learning models are determined by training a plurality of machine learning models, and by selecting the top performing model of the trained machine learning models.

In various embodiments, the plurality of machine learning models are automatically trained.

In various embodiments, the one or more machine learning models are independently any one of a random forest model, an extremely randomized tree (XRT) model, a generalized linear model (GLM), a gradient boosting machine (GBM), XGBoost, a stacked ensemble, and a deep learning algorithm (e.g., fully connected multi-layer artificial neural network).

In various embodiments, the one or more machine learning models are trained to predict site enrollment and site default likelihood for a specific disease indication.

In various embodiments, the one or more machine learning models achieve an AUC performance metric of at least 0.68 for predicting likelihood of a site default.

In various embodiments, the one or more machine learning models achieve a root mean squared error performance metric between 3.1-6.7 for predicting estimated number of enrolled patients at a site.

In various embodiments, the selected features comprise features associated with geographic locations, protocol complexity, study design, competitive landscape, and historical site enrollment metrics.

In various embodiments, the selected features comprise at least 3 of state of clinical trial site, study title, conditions, country, heading, sponsor, outcome measures, features associated with historical site enrollment metrics, investigator, and facility address.

In various embodiments, the selected features comprise at least 5 of state of clinical trial site, study title, conditions, country, heading, sponsor, outcome measures, features associated with historical site enrollment metrics, investigator, and facility address.

In various embodiments, the selected features comprise at least 10 of state of clinical trial site, study title, conditions, country, heading, sponsor, outcome measures, features associated with historical site enrollment metrics, investigator, and facility address.

In various embodiments, the features associated with historical site enrollment metrics comprise at least 3 of minimum, maximum, exponentially weighted moving average (EWMA), weighted average, and median of number of Enrolled patients for a trial, number of patients consented for a trial, number of patients completed a trial, number of patients that failed screening for a trial, and agility (e.g., time it took to start recruiting in a trial) over a reference time window at a reference entity.

In various embodiments, the features associated with historical site enrollment metrics comprise at least 5 of minimum, maximum, exponentially weighted moving average (EWMA), average, and median of number of Enrolled patients for a trial, number of patients consented for a trial, number of patients completed a trial, number of patients that failed screening for a trial, and agility.

In various embodiments, the features associated with historical site enrollment metrics comprise at least 10 of minimum, maximum, exponentially weighted moving average (EWMA), weighted average, and median of number of Enrolled patients for a trial, number of patients consented for a trial, number of patients completed a trial, number of patients that failed screening for a trial, and agility over a reference time window at a reference entity.

In various embodiments, the reference time is at least 1 year, at least 3 years, at least 5 years, or at least 10 years.

In various embodiments, the reference entity is one of a site, an investigator, a country, a state, a city, or a selected location.

In various embodiments, performing feature engineering on historical clinical trial data comprises converting trial metadata from the historical clinical trial data into a numerical representation of a single value or vector of values using n-grams, TFIDF, Word2vec, Glove, Fast Text, BERT, ELMo, InferSent.

In various embodiments, performing feature engineering on historical clinical trial data comprises applying a random forest feature selection algorithm to identify high importance features that have feature importance values above a threshold value.

In various embodiments, the historical clinical trial data is sourced from clinical trial databases selected from one or more of Data Query System (DQS), a contract research organization (CRO), Clinical Trial Management System (CTMS), or clinicaltrials.gov.

In various embodiments, the upcoming trial protocol comprises information for a clinical trial associated with any one of immunology, cardiovascular and metabolic diseases, infectious diseases, oncology, and neuroscience.

In various embodiments, the upcoming trial protocol comprises information for a clinical trial for any one of Crohn's Disease, Lupus, diabetic kidney disease, Lung Cancer, or respiratory syncytial virus (RSV).

In various embodiments, the predicted site enrollment comprises number of patients a site will enroll.

In various embodiments, the predicted site enrollment further comprises enrollment rate and/or agility, wherein the enrollment rate comprises number of patients per sit per month or year, and wherein the agility comprises time it took to start recruiting in a trial.

In various embodiments, the predicted site default likelihood comprises how likely a site is to enroll zero patients or fewer patients than a predetermined threshold.

Additionally disclosed herein is a non-transitory computer readable medium for determining or selecting one or more clinical trial sites for inclusion in a clinical trial, comprising instructions that, when executed by a processor, cause the processor to: obtain input data comprising data of an upcoming trial protocol; for each of the one or more clinical trial sites: generate a predicted site enrollment and a predicted site default likelihood for the clinical trial site by applying one or more machine learning models to selected features of the input data; rank the one or more clinical trial sites according to the predicted site enrollment and the predicted site default likelihood for the one or more clinical trial sites; and select top-ranked clinical trial sites, wherein each of the selected clinical trial sites has a predicted site enrollment above a first threshold value and a predicted site default likelihood below a second threshold value, wherein the selected features are previously determined by performing feature engineering on historical clinical trial data.

In various embodiments, the first threshold value is a median predicted site enrollment across the one or more clinical trial sites or a first specified value, and wherein the second threshold value is a median predicted site default likelihood across the one or more clinical trial sites or a second specified value.

In various embodiments, the non-transitory computer readable medium further comprises instructions that, when executed by the processor, cause the processor to visualize the predicted site enrollment and the predicted site default likelihood for the clinical trial sites in a quadrant graph.

In various embodiments, the non-transitory computer readable medium further comprises instructions that, when executed by the processor, cause the processor to generate a plurality of quantitative values informative of predicted enrollment timelines by applying a stochastic model to the predicted site enrollment and the predicted site default likelihood for the one or more clinical trial sites.

In various embodiments, the simulation method comprises Monte Carlo simulation.

In various embodiments, the plurality of quantitative values informative of predicted enrollment timeline comprises one or more of time to enroll between 50-1000 patients, number of patients enrolled in 4 months, number of patients enrolled in 12 months, number of patients enrolled in 18 months, number of patients enrolled in 24 months, and number of patients enrolled between 3-48 months.

In various embodiments, the predicted site enrollment and the predicted site default likelihood are validated by using one or more of the historical clinical trial data and prospective clinical trial data.

In various embodiments, the non-transitory computer readable medium further comprises an improvement of at least 11% in identifying the top-ranked clinical trial sites.

In various embodiments, the non-transitory computer readable medium further comprises instructions that, when executed by the processor, cause the processor to generate a site list of the selected top-ranked clinical trial sites.

In various embodiments, the site list comprises corresponding contact information useful for feasibility stakeholders.

In various embodiments, the one or more machine learning models are determined by training a plurality of machine learning models, and by selecting the top performing model of the trained machine learning models.

In various embodiments, the plurality of machine learning models are automatically trained.

In various embodiments, the one or more machine learning models are independently any one of a random forest model, an extremely randomized tree (XRT) model, a generalized linear model (GLM), a gradient boosting machine (GBM), XGBoost, a stacked ensemble, and a deep learning algorithm (e.g., fully connected multi-layer artificial neural network).

In various embodiments, the one or more machine learning models are trained to predict site enrollment and site default likelihood for a specific disease indication.

In various embodiments, the one or more machine learning models achieve an AUC performance metric of at least 0.68 for predicting likelihood of a site default.

In various embodiments, the one or more machine learning models achieve a root mean squared error performance metric between 3.1-6.7 for predicting estimated number of enrolled patients at a site.

In various embodiments, the selected features comprise features associated with geographic locations, protocol complexity, study design, competitive landscape, and historical site enrollment metrics.

In various embodiments, the selected features comprise at least 3 of state of clinical trial site, study title, conditions, country, heading, sponsor, outcome measures, features associated with historical site enrollment metrics, investigator, and facility address.

In various embodiments, the selected features comprise at least 5 of state of clinical trial site, study title, conditions, country, heading, sponsor, outcome measures, features associated with historical site enrollment metrics, investigator, and facility address.

In various embodiments, the selected features comprise at least 10 of state of clinical trial site, study title, conditions, country, heading, sponsor, outcome measures, features associated with historical site enrollment metrics, investigator, and facility address.

In various embodiments, the features associated with historical site enrollment metrics comprise at least 3 of minimum, maximum, exponentially weighted moving average (EWMA), weighted average, and median of number of Enrolled patients for a trial, number of patients consented for a trial, number of patients completed a trial, number of patients that failed screening for a trial, and agility (e.g., time it took to start recruiting in a trial) over a reference time window at a reference entity.

In various embodiments, the features associated with historical site enrollment metrics comprise at least 5 of minimum, maximum, exponentially weighted moving average (EWMA), average, and median of number of Enrolled patients for a trial, number of patients consented for a trial, number of patients completed a trial, number of patients that failed screening for a trial, and agility.

In various embodiments, the features associated with historical site enrollment metrics comprise at least 10 of minimum, maximum, exponentially weighted moving average (EWMA), weighted average, and median of number of Enrolled patients for a trial, number of patients consented for a trial, number of patients completed a trial, number of patients that failed screening for a trial, and agility over a reference time window at a reference entity.

In various embodiments, the reference time is at least 1 year, at least 3 years, at least 5 years, or at least 10 years.

In various embodiments, the reference entity is one of a site, an investigator, a country, a state, a city, or a selected location.

In various embodiments, the instructions that cause the processor to perform feature engineering on historical clinical trial data comprises converting trial metadata from the historical clinical trial data into a numerical representation of a single value or vector of values using n-grams, TFIDF, Word2vec, Glove, Fast Text, BERT, ELMo, InferSent.

In various embodiments, the instructions that cause the processor to perform feature engineering on historical clinical trial data comprises applying a random forest feature selection algorithm to identify high importance features that have feature importance values above a threshold value.

In various embodiments, the historical clinical trial data is sourced from clinical trial databases selected from one or more of Data Query System (DQS), a contract research organization (CRO), Clinical Trial Management System (CTMS), or clinicaltrials.gov.

In various embodiments, the upcoming trial protocol comprises information for a clinical trial associated with any one of immunology, cardiovascular and metabolic diseases, infectious diseases, oncology, and neuroscience.

In various embodiments, the upcoming trial protocol comprises information for a clinical trial for any one of Crohn's Disease, Lupus, diabetic kidney disease, Lung Cancer, or respiratory syncytial virus (RSV).

In various embodiments, the predicted site enrollment comprises number of patients a site will enroll.

In various embodiments, the predicted site enrollment further comprises enrollment rate and/or agility, wherein the enrollment rate comprises number of patients per sit per month or year, and wherein the agility comprises time it took to start recruiting in a trial.

In various embodiments, the predicted site default likelihood comprises how likely a site is to enroll zero patients or fewer patients than a predetermined threshold.

Additionally disclosed herein is a system for determining or selecting one or more clinical trial sites for inclusion in a clinical trial, comprising: a computer system configured to obtain input data comprising data of an upcoming trial protocol, wherein for each of the one or more clinical trial sites: the computer system generates a predicted site enrollment and a predicted site default likelihood for the clinical trial site by applying one or more machine learning models to selected features of the input data, wherein the computer system ranks the one or more clinical trial sites according to the predicted site enrollment and the predicted site default likelihood for the one or more clinical trial sites, wherein the computer system selects top-ranked clinical trial sites, wherein each of the selected clinical trial sites has a predicted site enrollment above a first threshold value and a predicted site default likelihood below a second threshold value, and wherein the selected features are previously determined by performing feature engineering on historical clinical trial data.

In various embodiments, the first threshold value is a median predicted site enrollment across the one or more clinical trial sites or a first specified value, and wherein the second threshold value is a median predicted site default likelihood across the one or more clinical trial sites or a second specified value.

In various embodiments, the system further comprises: an apparatus configured to visualize the predicted site enrollment and the predicted site default likelihood for the clinical trial sites in a quadrant graph.

In various embodiments, the computer system generates a plurality of quantitative values informative of predicted enrollment timelines by applying a stochastic model to the predicted site enrollment and the predicted site default likelihood for the one or more clinical trial sites.

In various embodiments, the simulation method comprises Monte Carlo simulation.

In various embodiments, the plurality of quantitative values informative of predicted enrollment timeline comprises one or more of time to enroll between 50-1000 patients, number of patients enrolled in 4 months, number of patients enrolled in 12 months, number of patients enrolled in 18 months, number of patients enrolled in 24 months, and number of patients enrolled between 3-48 months.

In various embodiments, the predicted site enrollment and the predicted site default likelihood are validated by using one or more of the historical clinical trial data and prospective clinical trial data.

In various embodiments, the system further comprises an improvement of at least 11% in identifying the top-ranked clinical trial sites.

In various embodiments, the computer system further generates a site list of the selected top-ranked clinical trial sites.

In various embodiments, the site list comprises corresponding contact information useful for feasibility stakeholders.

In various embodiments, the one or more machine learning models are determined by training a plurality of machine learning models, and by selecting the top performing model of the trained machine learning models.

In various embodiments, the plurality of machine learning models are automatically trained.

In various embodiments, the one or more machine learning models are independently any one of a random forest model, an extremely randomized tree (XRT) model, a generalized linear model (GLM), a gradient boosting machine (GBM), XGBoost, a stacked ensemble, and a deep learning algorithm (e.g., fully connected multi-layer artificial neural network).

In various embodiments, the one or more machine learning models are trained to predict site enrollment and site default likelihood for a specific disease indication.

In various embodiments, the one or more machine learning models achieve an AUC performance metric of at least 0.68 for predicting likelihood of a site default.

In various embodiments, the one or more machine learning models achieve a root mean squared error performance metric between 3.1-6.7 for predicting estimated number of enrolled patients at a site.

In various embodiments, the selected features comprise features associated with geographic locations, protocol complexity, study design, competitive landscape, and historical site enrollment metrics.

In various embodiments, the selected features comprise at least 3 of state of clinical trial site, study title, conditions, country, heading, sponsor, outcome measures, features associated with historical site enrollment metrics, investigator, and facility address.

In various embodiments, the selected features comprise at least 5 of state of clinical trial site, study title, conditions, country, heading, sponsor, outcome measures, features associated with historical site enrollment metrics, investigator, and facility address.

In various embodiments, the selected features comprise at least 10 of state of clinical trial site, study title, conditions, country, heading, sponsor, outcome measures, features associated with historical site enrollment metrics, investigator, and facility address.

In various embodiments, the features associated with historical site enrollment metrics comprise at least 3 of minimum, maximum, exponentially weighted moving average (EWMA), weighted average, and median of number of Enrolled patients for a trial, number of patients consented for a trial, number of patients completed a trial, number of patients that failed screening for a trial, and agility (e.g., time it took to start recruiting in a trial) over a reference time window at a reference entity.

In various embodiments, the features associated with historical site enrollment metrics comprise at least 5 of minimum, maximum, exponentially weighted moving average (EWMA), average, and median of number of Enrolled patients for a trial, number of patients consented for a trial, number of patients completed a trial, number of patients that failed screening for a trial, and agility.

In various embodiments, the features associated with historical site enrollment metrics comprise at least 10 of minimum, maximum, exponentially weighted moving average (EWMA), weighted average, and median of number of Enrolled patients for a trial, number of patients consented for a trial, number of patients completed a trial, number of patients that failed screening for a trial, and agility over a reference time window at a reference entity.

In various embodiments, the reference time is at least 1 year, at least 3 years, at least 5 years, or at least 10 years.

In various embodiments, the reference entity is one of a site, an investigator, a country, a state, a city, or a selected location.

In various embodiments, perform feature engineering on historical clinical trial data comprises converting trial metadata from the historical clinical trial data into a numerical representation of a single value or vector of values using n-grams, TFIDF, Word2vec, Glove, Fast Text, BERT, ELMo, InferSent.

In various embodiments, perform feature engineering on historical clinical trial data comprises applying a random forest feature selection algorithm to identify high importance features that have feature importance values above a threshold value.

In various embodiments, the historical clinical trial data is sourced from clinical trial databases selected from one or more of Data Query System (DQS), a contract research organization (CRO), Clinical Trial Management System (CTMS), or clinicaltrials.gov.

In various embodiments, the upcoming trial protocol comprises information for a clinical trial associated with any one of immunology, cardiovascular and metabolic diseases, infectious diseases, oncology, and neuroscience.

In various embodiments, the upcoming trial protocol comprises information for a clinical trial for any one of Crohn's Disease, Lupus, diabetic kidney disease, Lung Cancer, or respiratory syncytial virus (RSV).

In various embodiments, the predicted site enrollment comprises number of patients a site will enroll.

In various embodiments, the predicted site enrollment further comprises enrollment rate and/or agility, wherein the enrollment rate comprises number of patients per sit per month or year, and wherein the agility comprises time it took to start recruiting in a trial.

In various embodiments, the predicted site default likelihood comprises how likely a site is to enroll zero patients or fewer patients than a predetermined threshold.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “top-performing MLM 220A,” indicates that the text refers specifically to the element having that particular reference numeral. A reference numeral in the text without a following letter, such as “top-performing MLM 220,” refers to any or all of the elements in the figures bearing that reference numeral (e.g., “top-performing MLM 220” in the text refers to reference numerals “top-performing MLM 220A” and/or “top-performing MLM 220B” in the figures).

Figure (FIG. 1A depicts a system environment overview for generating a site prediction for use in determining or selecting sites for a clinical trial, in accordance with an embodiment.

FIG. 1B illustrates a block diagram of the site prediction system for use in determining or selecting sites for a clinical trial, in accordance with an embodiment.

FIG. 2A illustrates a block diagram for generating a site prediction for use in determining or selecting sites for a clinical trial, in accordance with an embodiment.

FIG. 2B illustrates a flow process for generating a site prediction for use in determining or selecting sites for a clinical trial, in accordance with an embodiment.

FIG. 3 illustrates a block diagram for performing training of a plurality of machine learning models, in accordance with an embodiment.

FIG. 4 illustrates an example computer for implementing the entities shown in FIG. 1A, 1B, 2A, 2B, 3.

FIG. 5 illustrates a first example of a site selection pipeline.

FIG. 6 illustrates a site selection pipeline overview.

FIG. 7 illustrates an example data ingestion process.

FIG. 8 illustrates example selected features by feature engineering.

FIG. 9 illustrates two example model performance for uses in model selection.

FIG. 10 illustrates an example model deployment process.

FIG. 11 illustrates an example visualization of site predictions for use in site evaluation.

FIG. 12 illustrates example clinical or therapeutic areas where the systems and methods in the presently disclosed embodiments can be applied.

FIG. 13A illustrates an example chart showing predictions from a machine learning model in a quadrant graph.

FIG. 13B is a chart illustrating example performance data associated with example execution of the site prediction system.

FIG. 14 illustrates additional example performance data associated with an example execution of the site prediction system.

FIG. 15 illustrates an example quadrant graph illustrating predictions from machine learning models.

FIG. 16 illustrates example data indicative of feature importance associated with an example execution of the site prediction system.

FIG. 17 illustrates a quadrant graph that visualizes data associated with an example execution of the site prediction system.

FIG. 18A illustrates a simulation process associated with the site prediction system.

FIG. 18B illustrates performance data associated with an example execution of the site prediction system.

FIG. 19 is an example graphical interface for displaying output data associated with the site prediction system.

### DETAILED DESCRIPTION

### I. System Environment Overview

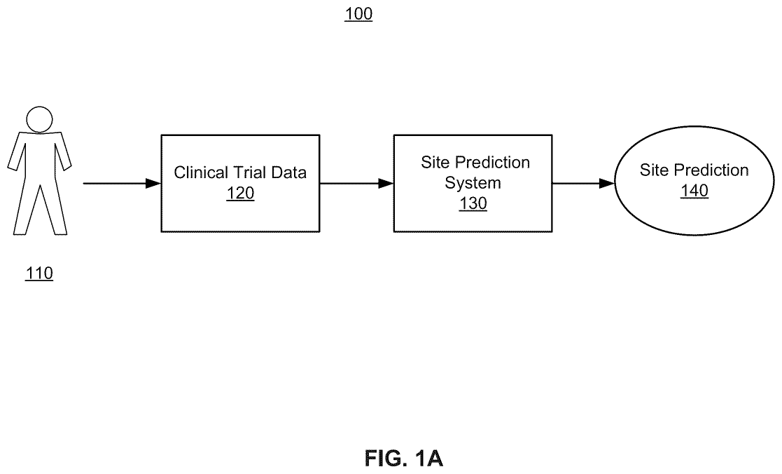

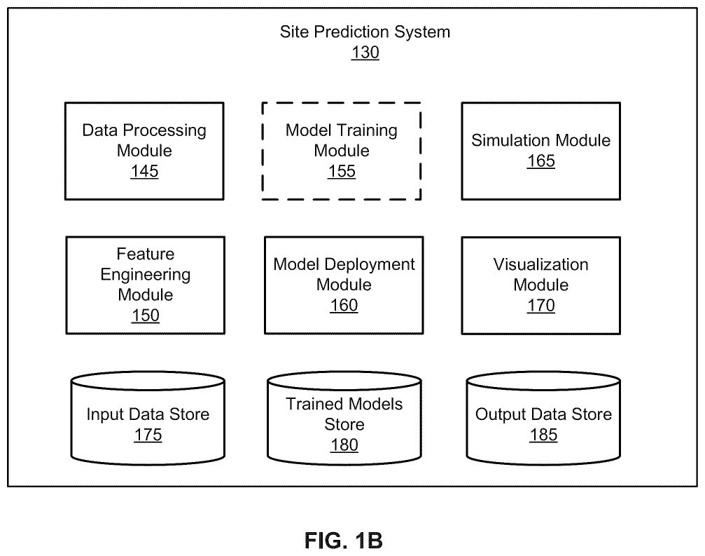

Figure (FIG. 1A depicts a system environment overview 100 for generating a site prediction for use in determining or selecting sites for a clinical trial, in accordance with an embodiment. The system environment 100 provides context in order to introduce a subject (or patient) 110, clinical trial data 120, and a site prediction system 130 for generating a site prediction 140.

The system environment 100 may include one or more subjects 110 who were enrolled in clinical trials that provide the clinical trial data 120. In various embodiments, a subject (or patient) may comprise a human or non-human, human or non-human, whether in vivo, ex vivo, or in vitro, male or female, a cell, tissue, or organism. In various embodiments, the subject 110 may have met eligibility criteria for enrollment in the clinical trials. For example, the subject 110 may have been previously diagnosed with a disease indication. Thus, the subject 110 may have been enrolled in a clinical trial that provides the clinical trial data 120 that tested a therapeutic intervention for treating the disease indication. Although FIG. 1A depicts one subject 110, in various embodiments, the system environment overview 100 may include two or more subjects 110 that were enrolled in clinical trials conducted by the clinical trial sites.

The clinical trial data 120 refers to clinical trial data related to one or more clinical trial sites and/or data of an upcoming trial protocol. In various embodiments, the clinical trial data 120 are related to one or more clinical trial sites that may have previously conducted a clinical trial (e.g., such that there are clinical operations data related to the previously conducted clinical trial). For example, the clinical trial sites where the clinical trial site data 120 is related to may have previously conducted one or more clinical trials that enrolled subjects 110. In various embodiments, the clinical trial site data 120 is related to one or more clinical trial sites that include at least one clinical facility and/or investigator that were previously used to conduct a clinical trial (e.g., in which the subjects 110 were enrolled) or can be used for one or more prospective clinical trials. In various embodiments, the clinical trial site data 120 is related to one or more clinical trial sites that are located in different geographical locations. In various embodiments, the clinical trial site data 120 is related to one or more clinical trial sites that generate or store clinical trial site date 120 describing the prior clinical trials (e.g., in which the subjects 110 were enrolled) that were conducted at the sites. In various embodiments, the clinical trial data 120 includes clinical operations data (e.g., clinical operation data that is not related to a subject 110) from one or more clinical trial sites. In various embodiments, the clinical trial data 120 includes site level enrollment data. In various embodiments, the clinical trial data 120 includes trial level enrollment data. In various embodiments, the clinical trial site data 120 is related to one or more clinical trial sites that were conducted for one or more different disease indications. Example disease indications are associated with any one of immunology, cardiovascular and metabolic diseases, infectious diseases, oncology, and neuroscience. In particular embodiments, the disease indication is any one of multiple myeloma, prostate cancer, non-small cell lung cancer, treatment resistant depression, Crohn's disease, systemic lupus erythematosus, hidradenitis suppurative/atopic dermatitis, diabetic kidney disease, or respiratory syncytial virus (RSV). In various embodiments, the clinical trial data 120 are data from one or more datasets related to an upcoming clinical trial dataset. For example, the clinical trial data 120 includes data of one or more protocols for an upcoming clinical trial related to a disease indication. Thus, the clinical trial data 120 related to one or more protocols for the upcoming clinical trial can be analyzed to predict likely top-performing sites that can be enrolled in the upcoming clinical trial.

In various embodiments, the clinical trial data 120 are obtained from internal clinical trial data, such as clinical trial data stored by a party operating the site prediction system 130. In various embodiments, the clinical trial data 120 are obtained from external clinical trial data, such as clinical trial data stored by a party different from the party operating the site prediction system 130. In various embodiments, the clinical trial data 120 are obtained from a combination of internal clinical trial data and external clinical trial data. In various embodiments, the clinical trial data 120 are obtained from one or more clinical trial sites In various embodiments, the clinical trial data 120 are obtained from a real-world database (e.g., a hospital). In various embodiments, the clinical trial data 120 are obtained from a public data set (e.g., a library).

The site prediction system 130 analyzes clinical trial data 120 and generates a site prediction 140. In particular embodiments, the site prediction system 130 generates a site prediction 140 for a specific disease indication that is to be treated in a future clinical trial, the site prediction 140 identifying the likely best performing clinical trial sites for the specific disease indication. In various embodiments, the site prediction system 130 applies one or more machine learning models and/or a stochastic model to analyze or evaluate clinical trial data 120 to generate the site prediction 140. In various embodiments, the site prediction system 130 includes or deploys one or more machine learning models that are trained using historical dataset from internal and/or external resources (e.g., industry sponsors and/or contract research organizations (CROs), etc.).

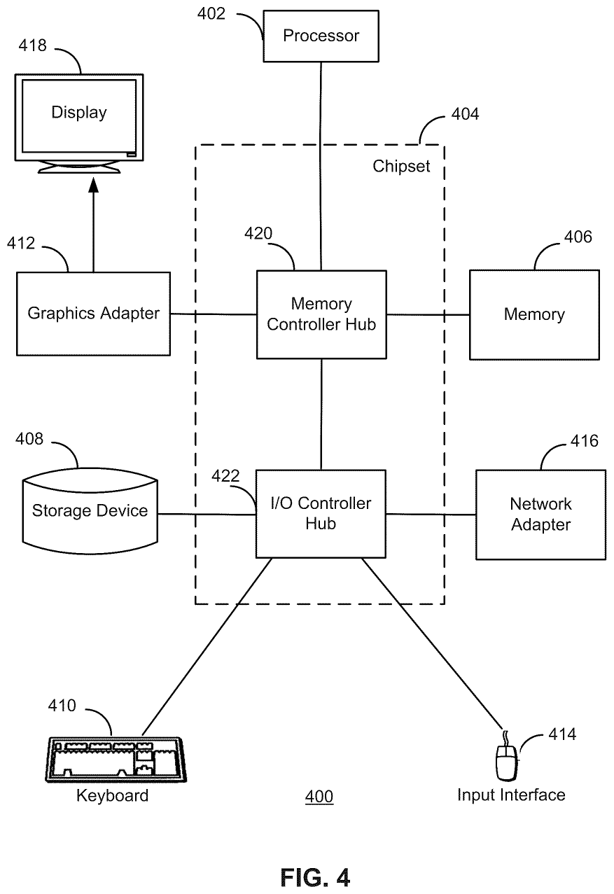

In various embodiments, the site prediction system 130 can include one or more computers, embodied as a computer system 400 as discussed below with respect to FIG. 4. Therefore, in various embodiments, the steps described in reference to the site prediction system 130 are performed in silico.

The site prediction 140 is generated by the site prediction system 130 and includes predictions of one or more clinical trial sites based on the clinical trial data 120 for selecting sites for a prospective clinical trial. In various embodiments, the site prediction system 130 may generate a site prediction 140 for each clinical trial site. For example, if there are X possible clinical trial sites that are undergoing site selection, the site prediction system 130 may generate a site prediction 140 for each of the X clinical trial sites. In various embodiments, X is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 50, at least 75, at least 100, at least 150, at least 200, at least 250, at least 300, at least 350, at least 400, at least 450, at least 500, at least 750, at least 1000, at least 1500, at least 2000, at least 2500, at least 3000, at least 3500, at least 4000, at least 4500, at least 5000, at least 5500, at least 6000, at least 6500, or at least 7000 clinical trial sites. In particular embodiments, X is at least 5000 clinical trial sites. In particular embodiments, X is at least 6000 clinical trial sites.

In various embodiments, the site prediction 140 includes a predicted site enrollment (e.g., number of patients a site will enroll) for one or more clinical trial sites involved in the clinical trial data 120. In various embodiments, the site prediction 140 includes a predicted site default likelihood (e.g., how likely a site is to enroll zero patients or fewer patients than a predetermined threshold) for one or more clinical trial sites involved in the clinical trial data 120. In various embodiments, the site prediction 140 includes a predicted site enrollment (e.g., number of patients a site will enroll) and a predicted site default likelihood (e.g., how likely a site is to enroll zero patients or fewer patients than a predetermined threshold) for one or more clinical trial sites involved in the clinical trial data 120. In various embodiments, in regards to the predicted site default likelihood, the predetermined threshold may be less than 100 patients, less than 75 patients, less than 50 patients, less than 40 patients, less than 30 patients, less than 20 patients, less than 15 patients, less than 10 patients, less than 9 patients, less than 8 patients, less than 7 patients, less than 6 patients, less than 5 patients, less than 4 patients, less than 3 patients, or less than 2 patients.

In various embodiments, the site prediction 140 includes predicted enrollment performance related to an enrollment timeline. For example, predicted enrollment performance related to an enrollment time may include a time to enroll a specific number of patients. As another example, predicted enrollment performance related to an enrollment time may include a predicted number of patients enrolled by a certain timepoint after enrollment begins.

In various embodiments, the site prediction 140 is or includes a list of ranked sites (e.g., sites that will enroll the highest number of patients) for a prospective clinical trial. In various embodiments, the site prediction 140 is or includes at least 5 of the top-ranked sites. In various embodiments, the site prediction 140 is or includes at least 10 of the top-ranked sites. In various embodiments, the site prediction 140 is or includes at least 20 of the top-ranked sites. In various embodiments, the site prediction 140 is or includes at least 50 of the top-ranked sites. In various embodiments, the site prediction 140 is or includes a list of the least ranked sites (e.g., lowest likelihood to enroll zero patients or fewer patients than a predetermined threshold) for a prospective clinical trial, such that the site prediction 140 enables a recipient of the list to avoid enrolling the lowest ranked sites for the prospective clinical trial. In various embodiments, the site prediction 140 is or includes at least 5 of the lowest-ranked sites. In various embodiments, the site prediction 140 is or includes at least 10 of the lowest-ranked sites. In various embodiments, the site prediction 140 is or includes at least 20 of the lowest-ranked sites. In various embodiments, the site prediction 140 is or includes at least 50 of the lowest-ranked sites. In various embodiments, the site prediction 140 can be transmitted to stakeholders so they can select sites for inclusion. In various embodiments, the site prediction 140 can be transmitted to principal investigators at the clinical trial site and/or stakeholders so they can determine whether to run the clinical trial at their site.

In various embodiments, the one or more clinical trial sites are categorized into tiers. For example, the one or more clinical trial sites can be categorized into a first tier representing the best performing clinical trial sites, a second tier representing the next best performing clinical trial sites, and so on. In various embodiments, the one or more clinical trial sites are categorized into four tiers. In various embodiments, the top tier of clinical trial sites are selected and included in a prediction e.g., a site prediction 140 shown in FIG. 1A that can be provided to appropriate stakeholders for inclusion in a subsequent clinical trial.

Reference is now made to FIG. 1B which depicts a block diagram illustrating the computer logic components of the site prediction system 130, in accordance with an embodiment. The components of the site prediction system 130 are hereafter described in reference to two phases. 1) a training phase and 2) a deployment phase. More specifically, the training phase refers to the building, developing, and training of models using training data. Therefore, the models are trained such that during the deployment phase, implementation of the models enables the generation of a site prediction (e.g., site prediction 140 in FIG. 1A). In some embodiments, both the steps performed during the training phase and the steps performed during the model deployment phase are performed by the site prediction system 130. In some embodiments, the steps performed during the model deployment phase are performed by the site prediction system 130, whereas the steps performed during the training phase are performed by a different party or system.

As shown in FIG. 1B, the site prediction system 130 includes a data processing module 145, a feature engineering module 150, a model training module 155, a model deployment module 160, a simulation module 165, a visualization module 170, an input data store 175, a trained models store 180, and an output data store 185. In various embodiments, the site prediction system 130 can be configured differently with additional or fewer modules. For example, a site prediction system 130 need not include the input data store 175. As another example, the site prediction system 130 need not include the model training module 155 (as indicated by the dotted lines in FIG. 1B), and instead, the model training module 155 is employed by a different system and/or party.

Generally, the data processing module 145 processes (e.g., ingests, cleans, integrates, enriches) the input data (e.g., clinical trial data 120 in FIG. 1A) stored in the input data store 175, and provides the processed data to the feature engineering module 150. Obtaining input data may include obtaining one or more clinical trial data from an external (e.g., publicly available) database or obtaining one or more clinical trial data from a locally available data store. In particular embodiments, obtaining input data involves obtaining historical clinical trial data. In particular embodiments, obtaining input data involves obtaining clinical trial data for a future clinical trial, such as an upcoming trial protocol. Obtaining one or more clinical trial data can encompass performing steps of pulling the one or more clinical trial data from the external (e.g., publicly available) database or the locally available data store. Obtaining input data can also encompass receiving one or more clinical trial data, e.g., from a party that has performed the steps of obtaining the one or more clinical trial data from the external (e.g., publicly available) database or the locally available data store. The one or more clinical trial data can be obtained by one of skill in the art via a variety of known ways including stored on a storage memory. In various embodiments, the input data can include locally available clinical trial data that are each pulled from a party at a single site. In such embodiments, the locally available clinical trial data is privately owned by the party at the single site.

The feature engineering module 150 extracts and selects features from the data processed by the data processing module 145. In various embodiments, the feature engineering module 150 provides extracted values of selected features to the model training module 155 for developing (e.g., training, validating, etc.) machine learning models. In various embodiments, the feature engineering module 150 provides extracted values of selected features to the model deployment module 160 for selecting top-performing machine learning models and for deploying the selected top-performing machine learned models to generate a site prediction (e.g., number of patients a site will enroll) and a predicted site default likelihood (e.g., how likely a site is to enroll zero patients or fewer patients than a predetermined threshold) for one or more clinical trial sites.

The model training module 155 develops (e.g., trains, validates, etc.) a plurality of machine learning models using selected features of the input data, and provides the trained machine learning models to the model deployment module 160. In various embodiments, a platform utilizing a proprietary framework or an open-source framework (e.g., H2O's AutoML framework) to automatically train and perform hyperparameter tuning on the plurality of machine learning models (e.g., generalized linear model (GLM), gradient boosting machine (GBM), XGBoost, stacked ensembles, deep learning, etc.). In various embodiments, the open-source framework may be scalable. The trained machine learning models may be locked and stored in the trained models store 180 to provide to the model deployment module 160 after the training is completed (e.g., until an quantitative improvement in the output of each model between each epoch or between each iteration of training is less than a pre-defined threshold, or until a maximum number of iterations is reached).

In various embodiments, the model deployment module 160 selects top-performing machine learning models and deploys the top-performing machine learning models. The model deployment module 160 may select top-performing machine learning models by evaluating or assessing the generated site predictions (e.g., a predicted site enrollment, a predicted site default likelihood, etc.). In various embodiments, the model deployment module 160 selects a best-performing machine learning model for each type of site prediction, based on the best training score as well as model interpretability. For example, the model deployment module 160 selects a best-performing machine learning model for predicting site enrollment, and a best-performing machine learning model for predicting site default likelihood. In various embodiments, the selected models for the site prediction variables are the same model. In various embodiments, the selected models for the site prediction variables are different models.

The model deployment module 160 implements the trained machine learned models stored in the trained models store 180 to analyze the values of selected features of the input data to generate site predictions such as a predicted site enrollment and a predicted site default likelihood. The model deployment module 160 provides the sites predictions generated from selected machine learning models to the simulation module 165.

In various embodiments, the machine learning models deployed by the model deployment module 160 can predict number of patients a clinical trial site will enroll in the next year. In various embodiments, the machine learning models deployed by the model deployment module 160 can predict number of patients a clinical trial site will enroll in the next 3 years. In various embodiments, the machine learning models deployed by the model deployment module 160 can predict number of patients a clinical trial site will enroll in the next 5 years. In various embodiments, the machine learning models deployed by the model deployment module 160 can predict number of patients a clinical trial site will enroll within a M time period. In various embodiments, M is any of 6 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years. In various embodiments, M can be any number.

In various embodiments, the machine learning models deployed by the model deployment module 160 can predict how likely a site is to enroll zero patients or fewer patients than a predetermined threshold in the next year. In various embodiments, the machine learning models deployed by the model deployment module 160 can predict how likely a site is to enroll zero patients or fewer patients than a predetermined threshold in the next 3 years. In various embodiments, the machine learning models deployed by the model deployment module 160 can predict how likely a site is to enroll zero patients or fewer patients than a predetermined threshold in the next 5 years. In various embodiments, the machine learning models deployed by the model deployment module 160 can predict how likely a site is to enroll zero patients or fewer patients than a predetermined threshold within a M time period. In various embodiments, M is any of 6 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years. In various embodiments, M can be any number. In various embodiments, the predetermined threshold may be less than 100 patients, less than 75 patients, less than 50 patients, less than 40 patients, less than 30 patients, less than 20 patients, less than 15 patients, less than 10 patients, less than 9 patients, less than 8 patients, less than 7 patients, less than 6 patients, less than 5 patients, less than 4 patients, less than 3 patients, or less than 2 patients.

The simulation module 165 applies a stochastic model (e.g., Monte Carlo simulation) using the site predictions generated from selected machine learning models, as input, to generate enrollment timeline prediction 245 (e.g., multi-site enrollment timelines). Example descriptions of a Monte Carlo simulation are found in Abbas I. et al.: Clinical trial optimization: Monte Carlo simulation Markov model for planning clinical trials recruitment, Contemporary Clinical Trials 28:220-231, 2007, which is hereby incorporated by reference in its entirety.

The visualization module 170 generates a visualization of the predictions generated by deploying top-performing machine learning models using the model deployment module 160 and/or by the stochastic model using the simulation module 165. In various embodiments, the visualization module 170 generates a visualization of the predicted site enrollment 225 and of the predicted site default likelihood 230 for the clinical trials sites generated by the top-performing models by the model deployment module 160. For example, the visualization module 170 may present the predicted site enrollment 225 and the predicted site default likelihood 230 in a quadrant graph. In various embodiments, the visualization module 170 generates a visualization of the enrollment timeline prediction 245 generated by the stochastic model 240 in a graph that includes statistically-robust enrollment curves. Examples of visualizations are shown in 8-19 described below in the context of specific examples. Similar visualizations may be generated in relation to other executions of the site prediction system 130.

The input data store 175 stores clinical trial data (e.g., clinical trial data 120 in FIG. 1A). In various embodiments, at least some of the clinical trial data stored in the input data store 175 are used for training machine learning models. In various embodiments, at least some of the clinical trial data stored in the input data store 175 are used for implementing trained machine learning models. In various embodiments, the clinical trial data used for training machine learning models include a same clinical trial site as the clinical trial data used for implementing the trained machine learning models In various embodiments, the clinical trial data used for training machine learning models include a different clinical trial site from the clinical trial data used for implementing the trained machine learning models.

The trained models store 180 stores trained machine learning models (e.g., GLM, GBM, XGBoost, stacked ensembles, deep learning, etc.) for selection and implementation in the deployment phase.

The output data store 185 stores the site predictions (e.g., site predictions 140 in FIG. 1A) generated by selected top-performing machine learning models using the model deployment module 160 and by the stochastic model using the simulation module 165. In various embodiments, the output data store 185 stores additional insights coming from the machine learning models, such as feature importance, most important features, and/or the impact of each feature (e.g., Shapley Additive Explanations (SHAP) values) for each variable in the prediction, model performance metrics (e.g., AUC, RMSE, etc.). In various embodiments, the data and/or additional insights stored in the output data store 185 can be provided to the visualization module 170 for visualizing site predictions. In various embodiments, the data and/or additional insights stored in the output data store 185 can be provided to appropriate stakeholders for site selection or determination in an upcoming clinical trial.

In various embodiments, the components of the site prediction system 130 are applied during one of the training phase and the deployment phase. For example, the model training module 155 are applied during the training phase to train a model. Additionally, the model deployment module 160 is applied during the deployment phase. In various embodiments, the components of the site prediction system 130 can be performed by different parties depending on whether the components are applied during the training phase or the deployment phase. In such scenarios, the training and deployment of the prediction model are performed by different parties. For example, the model training module 155 and training data applied during the training phase can be employed by a first party (e.g., to train a model) and the model deployment module 160 applied during the deployment phase can be performed by a second party (e.g., to deploy the model). Training models and deploying models are described in further detail below.

### II. Methods for Generating Site Prediction

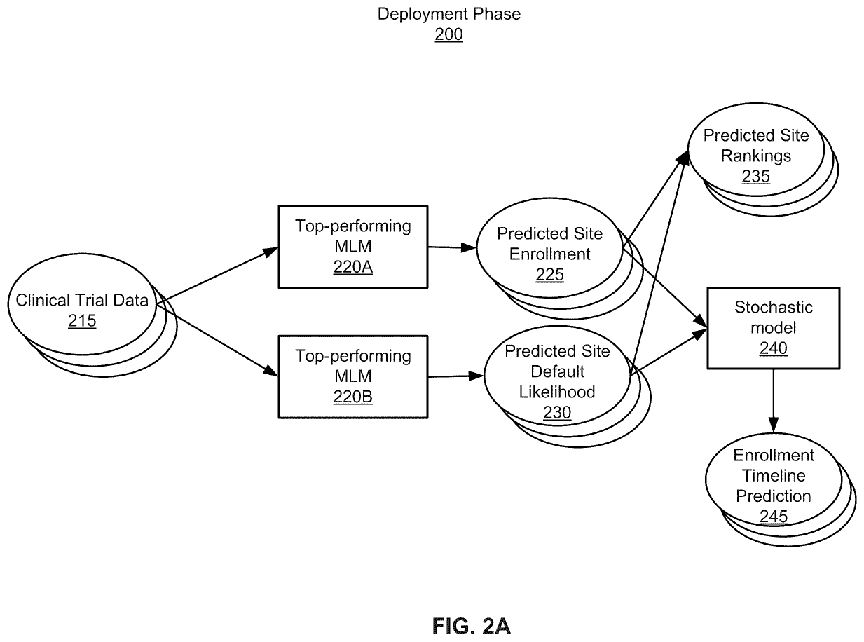

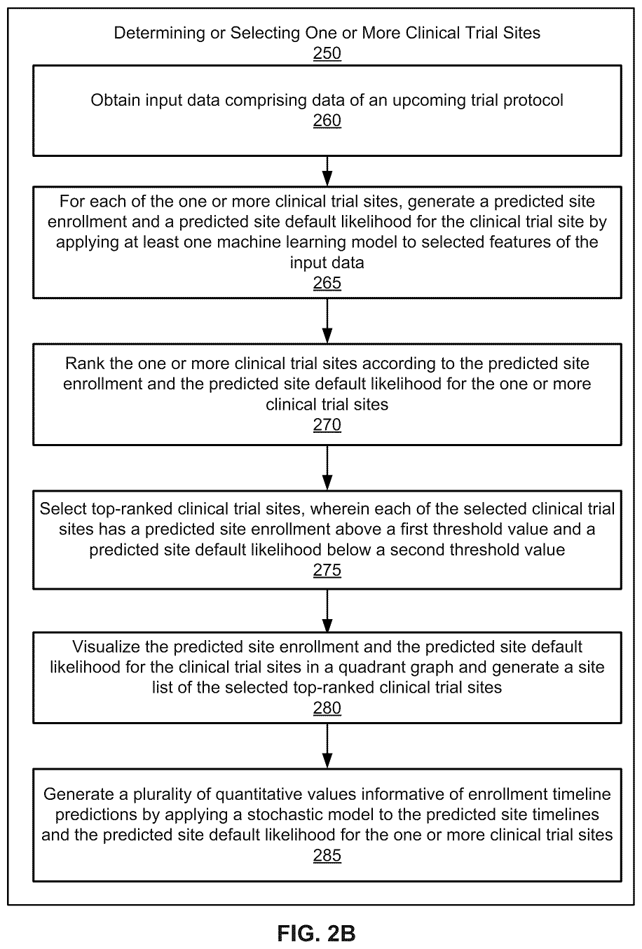

Embodiments described herein include methods for generating a site prediction for one or more clinical trial sites by applying one or more trained models to analyze selected features of the input data related to the one or more clinical trial sites. Such methods can be performed by the site prediction system 130 described in FIG. 1B. Reference will further be made to FIG. 2A, which depicts an example block diagram for generating a site prediction for uses such as site selection, determination, or planning, in accordance with an embodiment.

As shown in FIG. 2A, the deployment phase 200 begins with obtaining clinical trial data 215. In various embodiments, the clinical trial data 215 comprises upcoming trial protocol information associated with a future clinical trial. For example, the future clinical trial may be designed for a particular disease indication. Therefore, the upcoming trial protocol information can include information associated with the particular disease indication, such as any one of immunology, cardiovascular and metabolic diseases, infectious diseases, oncology, and neuroscience. In particular embodiments, the upcoming trial protocol includes information for a clinical trial for any one of Crohn's disease, lupus, diabetic kidney disease, lung cancer, or respiratory syncytial virus (RSV). In various embodiments, the clinical trial data 215 includes historical clinical trial data derived from one or more clinical trial sites. Here, the historical clinical trial data may be a subset of clinical trial data 120 described in FIG. 1A) and may be stored in an input data store (e.g., input data store 175 in FIG. 1B). In various embodiments, the historical clinical trial data is obtained from one or more resources (e.g., internal database, external industry sponsor, publicly available database, hospital, etc.). In particular embodiments, the historical clinical trial data is obtained or sourced from clinical trial databases selected from one or more of Data Query System (DQS), a contract research organization (CRO), Clinical Trial Management System (CTMS), or clinicaltrials.gov. In various embodiments, the clinical trial data 215 includes upcoming trial protocol information and does not include historical clinical trial data. In various embodiments, the clinical trial data 215 includes upcoming trial protocol information and further includes historical clinical trial data.

As shown in FIG. 2A, the clinical trial data 215 can be directly provided as input to one or more machine learning models, such as top-performing MLM 220A and top-performing MLM 220B. In some embodiments, the clinical trial data 215 can undergo processing and/or feature extraction (e.g., by the feature engineering module 150 in FIG. 1B) prior to being provided as input to the one or more machine learning models. In various embodiments, the clinical trial data 215 undergoes processing (e.g., cleaned, integrated, and enriched) using the data processing module 145, as described herein In various embodiments, the clinical trial data 215 undergoes feature extraction (e.g., by feature engineering module 150), based on selected features that were previously determined. The selecting of features is described in further detail herein.

In various embodiments, the selected features may include features associated with geographic locations, protocol complexity, study design, competitive landscape, and historical site enrollment metrics. In particular embodiments, the selected features include state of clinical trial site, study title, conditions, country, heading, sponsor, outcome measures, features associated with historical site enrollment metrics, investigator, and/or facility address. In particular embodiments, the selected features associated with historical site enrollment metrics include statistical measures, such as any of a minimum, maximum, exponentially weighted moving average (EWMA), weighted average, and median values. In particular embodiments, the selected features associated with historical site enrollment metrics includes at least 1, 3, 5, or 10 of minimum, maximum, exponentially weighted moving average (EWMA), weighted average, and median of number of enrolled patients for a trial, number of patients consented for a trial, number of patients completed a trial, number of patients that failed screening for a trial, and agility (e.g., time it took to start recruiting in a trial) over a reference time window at a reference entity. In particular embodiments, the reference time is at least 1 year, at least 3 years, at least 5 years, or at least 10 years. In various embodiments, the reference time can be any time period. In particular embodiments, the reference entity is one of a site, an investigator, a country, a state, a city, or a selected location.

As shown in FIG. 2A, the selected features of the clinical trial data 215, are provided as input for implementing one or more top-performing machine learning models (MLMs) 220A and 220B to generate site predictions (e.g., part of the site prediction 140 in FIG. 1A) that include predicted site enrollment 225 and predicted site default likelihood 230.

In various embodiments, the top performing MLMs 220A and 220B were previously trained using training data, as is described in further detail herein. In various embodiments, the training data can be historical trial data. In various embodiments, the top-performing MLMs 220A and 220B were previously determined by training a plurality of machine learning models, and by selecting the top performing model of the trained machine learning models to predict site enrollment (e.g., number of patients a site will enroll) and/or site default likelihood (e.g., how likely a site is to enroll zero patients or fewer patients than a predetermined threshold) for a specific disease indication. For example, the top-performing MLM 220A may be the best-performing MLM for generating predicted site enrollment 225, and the top-performing MLM 220B may be the best-performing MLM for generating site default likelihood 230. In various embodiments, the top performing MLMs 220A and 220B are constructed as a single model. For example, MLMs 220A and 220B are constructed as single model, which outputs predicted site enrollment 225 and predicted site default likelihood 230. In various embodiments, the top performing MLMs 220A and 220B are separate models. In various embodiments, the top performing MLMs 220A or 220B are independently any one of a random forest model, an extremely randomized trees (XRT) model, a generalized linear model (GLM), a gradient boosting machine (GBM), XGBoost, a stacked ensemble, and a deep learning algorithm (e.g., fully connected multi-layer artificial neural network). In various embodiments, MLM 220A is a regression model that predicts a continuous value representing the predicted site enrollment 225. In various embodiments, MLM 220B is a classifier that predicts a classification representing the predicted site default likelihood 230 (e.g., default or no default).

In various embodiments, the predicted site enrollment 225 represents a “number enrolled” variable, and the predicted site default likelihood 230 represents a “site default” variable. In various embodiments, a “site default” variable that is equal to zero refers to a site that enrolled more than one patient, and thus the site has not defaulted. In various embodiments, a “site default” variable that is equal to 1 refers to a site that enrolled zero or one patient, and thus the site has defaulted. In various embodiments, the “number enrolled” variable refers to number of patients enrolled at a site.

In various embodiments, the predicted site enrollment 225 includes enrollment rate (e.g., number of patients per sit per month/year) and/or agility (time required for a site to start up and begin recruitment).

In various embodiments, the predicted site enrollment 225 and predicted site default likelihood 230 are validated by using one or more of the historical clinical trial data and/or prospective clinical trial data.

The predicted site enrollment 225 and predicted site default likelihood 230 can be used to generate predicted site rankings 235. In various embodiments, the predicted site enrollment 225 and predicted site default likelihood 230 are compared to one or more threshold values to generate predicted site rankings 235. For example, the predicted site enrollment 225 for a site can be compared to a first threshold value and the predicted site default likelihood 230 for a site can be compared to a second threshold value. Generally, a site that has a predicted site enrollment that is above the first threshold value and a predicted site default likelihood that is below the second threshold value will be ranked more highly than another site in which either the predicted site enrollment is below the first threshold or the predicted site default likelihood is above the second threshold.

In various embodiments, the first threshold value and the second threshold values are statistical measures. A statistical measure can be a mean value, a median value, or a mode value. For example, the first threshold value can be the median site enrollment across historical data of all clinical trial sites or a specified value (e.g., a value in the top-performing quadrant or quartile). The second threshold value can be the median predicted site default likelihood across historical data of all clinical trial sites or a specified value (e.g., a value in the low-performing quadrant or quartile). In various embodiments, the first threshold value and the second threshold value are fixed values. For example, the first threshold value may be a fixed value of at least 1 enrolled patient, at least 2 enrolled patients, at least 3 enrolled patient, at least 4 enrolled patient, at least 5 enrolled patients, at least 6 enrolled patients, at least 7 enrolled patients, at least 8 enrolled patients, at least 9 enrolled patients, at least 10 enrolled patients, at least 15 enrolled patients, at least 20 enrolled patients, at least 25 enrolled patients, at least 30 enrolled patients, at least 35 enrolled patients, at least 40 enrolled patients, at least 50 enrolled patients, at least 75 enrolled patients, at least 100 enrolled patients, at least 200 enrolled patients, at least 300 enrolled patients, at least 400 enrolled patients, at least 500 enrolled patients, or at least 1000 enrolled patients. As another example, the second threshold value may be a fixed value of less than 30% likelihood of default, less than 25% likelihood of default, less than 20% likelihood of default, less than 15% likelihood of default, less than 14% likelihood of default, less than 13% likelihood of default, less than 12% likelihood of default, less than 11% likelihood of default, less than 10% likelihood of default, less than 9% likelihood of default, less than 8% likelihood of default, less than 7% likelihood of default, less than 6% likelihood of default, less than 5% likelihood of default, less than 4% likelihood of default, less than 3% likelihood of default, less than 2% likelihood of default, or less than 1% likelihood of default.

In various embodiments, the predicted site rankings 235 is a list of all ranked sites. In various embodiments, the predicted site rankings 235 is a list of selected top-ranked clinical trial sites. In various embodiments, each of the top-ranked clinical trial sites included in the predicted site rankings 235 has a predicted site enrollment above a first threshold value and a predicted site default likelihood below a second threshold value.

In various embodiments, the predicted site rankings 235 is a list of at least 3 top-ranked clinical trial sites. In various embodiments, the predicted site rankings 235 is a list of at least 5 top-ranked clinical trial sites. In various embodiments, the predicted site rankings 235 is a list of at least 10 top-ranked clinical trial sites. In various embodiments, the predicted site rankings 235 is a list of at least 20 top-ranked clinical trial sites. In various embodiments, the predicted site rankings 235 is a list of at least 50 top-ranked clinical trial sites. In various embodiments, the predicted site rankings 235 includes corresponding contact information useful for feasibility stakeholders, such as address, country, investigator, contact information, and other suitable information of each site listed in the predicted site rankings 235.

The predicted site enrollment 225 and predicted site default likelihood 230 can be used as an input to a stochastic model 240 (e.g., Monte Carlo simulation) to generate a plurality of quantitative values informative of enrollment timeline predictions 245.

In various embodiments, the plurality of quantitative values informative of enrollment timeline prediction 245 includes time to enroll a number of patients. In various embodiments, the plurality of quantitative values informative of enrollment timeline prediction 245 includes time to enroll at least 5 patients. In various embodiments, the plurality of quantitative values informative of enrollment timeline prediction 245 includes time to enroll at least 10 patients. In various embodiments, the plurality of quantitative values informative of enrollment timeline prediction 245 includes time to enroll at least 50 patients. In various embodiments, the plurality of quantitative values informative of enrollment timeline prediction 245 includes time to enroll at least 100 patients. In various embodiments, the plurality of quantitative values informative of enrollment timeline prediction 245 includes time to enroll at least 500 patients. In various embodiments, the plurality of quantitative values informative of enrollment timeline prediction 245 includes time to enroll at least 1000 patients. In various embodiments, the plurality of quantitative values informative of enrollment timeline prediction 245 includes time to enroll at least 2000 patients. In various embodiments, the plurality of quantitative values informative of enrollment timeline prediction 245 includes time to enroll a range of 50-1000 patients.

In various embodiments, the plurality of quantitative values informative of enrollment timeline prediction 245 includes number of patients enrolled in a time period. In various embodiments, the plurality of quantitative values informative of enrollment timeline prediction 245 includes number of patients enrolled in 1 month. In various embodiments, the plurality of quantitative values informative of enrollment timeline prediction 245 includes number of patients enrolled in 4 month. In various embodiments, the plurality of quantitative values informative of enrollment timeline prediction 245 includes number of patients enrolled in 6 month. In various embodiments, the plurality of quantitative values informative of enrollment timeline prediction 245 includes number of patients enrolled in 12 month In various embodiments, the plurality of quantitative values informative of enrollment timeline prediction 245 includes number of patients enrolled in 18 month. In various embodiments, the plurality of quantitative values informative of enrollment timeline prediction 245 includes number of patients enrolled in 24 month. In various embodiments, the plurality of quantitative values informative of enrollment timeline prediction 245 includes number of patients enrolled in a range of 18-24 months. In various embodiments, the plurality of quantitative values informative of enrollment timeline prediction 245 includes number of patients enrolled in a range of 3-48 months.

In particular embodiments, the plurality of quantitative values informative of predicted enrollment performance comprises one or more of time to enroll 500 patients, number of patients enrolled in 4 months, number of patients enrolled in 12 months, number of patients enrolled in 18 months, or number of patients enrolled in 24 months.

Reference is now made to FIG. 2B, which depicts a flow process 250 for deploying models for uses in determining or selecting one or more clinical trial sites, in accordance with an embodiment.

At step 260, input data comprising data of an upcoming trial protocol is obtained.

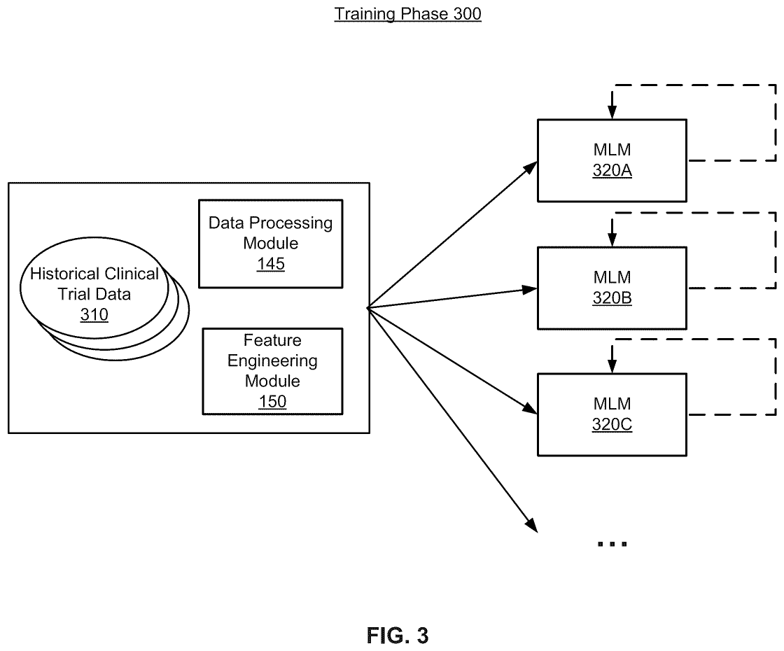

At step 265, for each of one or more clinical trial sites, one or more machine learning models (e.g., top-performing MLM 220A and 220B) are applied to selected features of the input data to generate a predicted site enrollment and a predicted site default likelihood for the clinical trial site. In various embodiments, the selected features are previously determined by performing feature engineering on historical clinical trial data (e.g., historical clinical trial data 310 in FIG. 3). In various embodiments, here at step 265, features of the input data can be engineered or extracted and can be provided as input to the one or more machine learning models.

At step 270, the one or more clinical trial sites are ranked according to the predicted site enrollment and the predicted site default likelihood for the one or more clinical trial sites.

At step 275, top-ranked clinical trial sites are selected from the ranked clinical trial sites. In various embodiments, each of the selected clinical trial sites has a predicted site enrollment above a first threshold value and a predicted site default likelihood below a second threshold value. In various embodiments, the first threshold value is a median predicted site enrollment across the one or more clinical trial sites. In various embodiments, the second threshold value is a median predicted site default likelihood across the one or more clinical trial sites.

At step 280, the predicted site enrollment and the predicted site default likelihood for the ranked clinical trial sites are visualized in a quadrant graph, and a site list of the selected top-ranked clinical trial sites is generated. The quadrant graph and/or the site list can be evaluated or provided to appropriate stakeholders for determining or selecting in an upcoming clinical trial.

At step 285, a plurality of quantitative values informative of enrollment timeline prediction is generated by applying a stochastic model (e.g., stochastic model 240 in FIG. 2A) to the predicted site enrollment and the predicted site default likelihood for the one or more clinical trial sites. The plurality of quantitative values informative of enrollment timeline prediction can be evaluated or provided to appropriate stakeholders for determining or selecting in an upcoming clinical trial.

III. Training a Machine Learning Model for deployment in a Site Prediction System

FIG. 3 depicts an example block diagram for a training phase 300, in accordance with an embodiment. In various embodiments, the training phase 300 is included in the site prediction system 130. In various embodiments, the training phase 300 is not included in the site prediction system 130, but is conducted in another system or by another party. For example, the training phase 300 can be conducted by a proprietary framework or an open-source framework (e.g., H2O's AutoML framework) to automatically train and perform hyperparameter tuning on a large selection of machine learning models (e.g., GLM, GBM, XGBoost, stacked ensembles, deep learning, etc.)

As shown in FIG. 3, the training phase 300 includes training data, such as historical clinical trial data 310, the data processing module 145 and feature engineering module 150 for processing and analyzing the historical clinical trial data 310, and a plurality of machine learning models (MLMs) 320A, 320B, 320C, etc., which are trained during the training phase 300.

In various embodiments, the historical clinical trial data 310 may be a subset of the input data (e.g., clinical trial data 120 in FIG. 1A) and stored in an input data store (e.g., input data store 175 in FIG. 1B). In various embodiments, the historical clinical trial data 310 is obtained from one or more resources (e.g., internal database, external industry sponsor, publicly available database, hospital, etc.). In particular embodiments, the historical clinical trial data 310 is obtained or sourced from clinical trial databases selected from one or more of Data Query System (DQS), a contract research organization (CRO), Clinical Trial Management System (CTMS), or clinicaltrials.gov.

In various embodiments, the historical clinical trial data 310 includes site level enrollment data and/or trial level data of a historical clinical trial. For example, the historical clinical trial data 310 include enrollment number per site, default status (e.g., 0 or 1 patients were enrolled), enrollment rate (e.g., number of patients per sit per month/year), enrollment dates such as agility (e.g., time required for a site to start up and begin recruitment) or enrollment period, etc., investigator names, site locations, trial sponsor, list of trial identifiers for disease indication, eligibility criteria, protocol information, trial dates (e.g., start date, end date, etc.), and/or site ready time of a historical clinical trial.

The historical clinical trial data 310 is processed (e.g., cleaned, integrated, and enriched) using the data processing module 145. In various embodiments, the data processing module 145 clean the historical clinical trial data 310 by assessing each column of the historical clinical trial data 310, followed by cleaning methods such as standardizing date formats, removing null values, removing new line characters, cleaning column names, parsing or cleaning age criteria, and other appropriate cleaning steps.

In various embodiments, the data processing module 145 integrates the cleaned historical clinical trial data 310 by merging datasets of the historical clinical trial data 310 based on the National Clinical Trial (NCT) number. The data processing module 145 may perform the integration and merging of datasets if the historical clinical trial data 310 includes multiple datasets that are obtained from multiple databases. In various embodiments, the cleaned historical clinical trial data 310 is integrated so that each row includes trial performance for each site-investigator-pair. In various embodiments, the cleaned historical clinical trial data 310 is integrated so that there are multiple rows for each trial. In various embodiments, the cleaned historical clinical trial data 310 is integrated so that there are multiple rows for each site-investigator pair. In various embodiments, the cleaned historical clinical trial data 310 is integrated so that there is a unique row for each site-investigator performance for a given trial.

In various embodiments, the data processing module 145 enriches the cleaned and integrated historical clinical trial data 310 by splitting inclusion and exclusion criteria, and/or standardizing names.

Generally, the feature engineering module 150 extracts features that are related to facilities or investigators of the processed historical clinical trial data 310, and selects top features by applying a random forest feature selection algorithm to identify high importance features that have feature importance values above a threshold value. In various embodiments, the feature engineering module 150 extracts features by converting or transforming tagged trial metadata (e.g., text, words) from the historical clinical trial data 310 into a numerical representation of a single value or vector of values using n-grams, TFIDF, Word2vec, Glove, Fast Text, BERT, ELMo, InferSent. In various embodiments, the feature engineering module 150 extracts time series features that capture historical performance of a site in the past M time period.

In various embodiments, the selected features may include features associated with geographic locations, protocol complexity, study design, competitive landscape, and historical site enrollment metrics. In particular embodiments, the selected features include state of clinical trial site, study title, conditions, country, heading, sponsor, outcome measures, features associated with historical site enrollment metrics, investigator, and/or facility address. In particular embodiments, the selected features associated with historical site enrollment metrics includes at least 1, 3, 5, or 10 of minimum, maximum, exponentially weighted moving average (EWMA), weighted average, and median number of enrolled patients for a trial, number of patients consented for a trial, number of patients completed a trial, number of patients that failed screening for a trial, and agility (e.g., time it took to start recruiting in a trial) over a reference time window at a reference entity. In particular embodiments, the reference time is at least 1 year, at least 3 years, at least 5 years, or at least 10 years. In particular embodiments, the reference entity is one of a site, an investigator, a country, a state, a city, or a selected location.

As a specific example, given a reference time window of 2 years and a reference entity of a site, example resulting features include:
Max/Min/EWMA/mean/median number of enrolled patients for a trial in the last 2 years at the respective site
Max/Min/EWMA/mean/median number of patients consented for a trial in the last 2 years at the respective site
Max/Min/EWMA/mean/median number of patients completed a trial in the last 2 years at the respective site
Max/Min/EWMA/mean/median number of patients that failed screening for a trial in the last 2 years at the respective site
Max/Min/EWMA/mean/median agility (time it took to start recruiting in a trial) in the last 2 years at the respective site

As another specific example, given a reference time window of 1 year and a reference entity of an investigator, example resulting features include:
Max/Min/EWMA/mean/median number of Enrolled patients for a trial in the last year at the respective investigator
Max/Min/EWMA/mean/median number of patients consented for a trial in the last year at the respective investigator
Max/Min/EWMA/mean/median number of patients completed a trial in the last year at the respective investigator
Max/Min/EWMA/mean/median number of patients that failed screening for a trial in the last year at the respective investigator
Max/Min/EWMA/mean/median agility (time it took to start recruiting in a trial) in the last year at the respective investigator

As another specific example, given a reference time window of 5 years and a reference entity of a country, example resulting features include:
Max/Min/EWMA/mean/median number of Enrolled patients for a trial in the last 5 years at the respective country
Max/Min/EWMA/mean/median number of patients consented for a trial in the last 5 years at the respective country
Max/Min/EWMA/mean/median number of patients completed a trial in the last 5 years at the respective country
Max/Min/EWMA/mean/median number of patients that failed screening for a trial in the last 5 years at the respective country
Max/Min/EWMA/mean/median agility (time it took to start recruiting in a trial) in the last 5 years at the respective country

In various embodiments, the feature engineering module 150 extracts or selects at least 3 features. In various embodiments, the feature engineering module 150 extracts or selects at least 5 features. In various embodiments, the feature engineering module 150 extracts or selects at least 10 features. In various embodiments, the feature engineering module 150 extracts or selects at least 50 features. In various embodiments, the feature engineering module 150 extracts or selects at least 100 features. In various embodiments, the feature engineering module 150 extracts or selects at least 500 features. In various embodiments, the feature engineering module 150 extracts or selects at least 1000 features. In various embodiments, the feature engineering module 150 extracts or selects at least 2000 features. In particular embodiments, the feature engineering module 150 extracts or selects at least 1700 features.

The model training module 155 trains the plurality of machine learning models (MLMs) 320A, 320B, 320C, etc. by providing the extracted features of the historical clinical trial data 310 as input. As shown in FIG. 3, as indicated by the dotted lines, the output values of MLMs 320A, 320B, 320C, etc. may be used to train each respective model. In various embodiments, each of MLMs 320A, 320B, 320C, etc. is individually trained. Specifically, the output value of MLM 320A is used to further train MLM 320A. The output value of MLM 320B is used to further train MLM 320B. The output value of MLM 320C is used to further train MLM 320C. For example, each of MLMs 320A, 320B, 320C, etc. can be individually and iteratively trained until an quantitative improvement in the output of each model between each epoch or between each iteration of training is less than a pre-defined threshold, or until a maximum number of iterations is reached. Each of MLMs 320A, 320B, 320C, etc. may be locked after the training is completed, and a training score for each of MLMs 320A, 320B, 320C, etc. may be used during model assessment to determine or select the top-performing models (e.g., top-performing MLM 220A and 220B in FIG. 2A).

In various embodiments, one or more of MLMs 320A, 320B, 320C, etc. are individually trained to minimize a loss function such that the output of each model is improved over successive training epochs. In various embodiments, the loss function is constructed for any of a least absolute shrinkage and selection operator (LASSO) regression, Ridge regression, or ElasticNet regression. In such embodiments, the dotted lines for the models shown in FIG. 3 can represent the backpropagation of a loss value calculated based on the loss function. Thus, one or more of the models are trained based on the backpropagated value such that the model improves its predictive capacity.

### IV. Example Machine Learning Models

Generally, a machine learning model (MLM) is structured such that it analyzes input data or extracted features of input data associated with a clinical trial site and/or an upcoming trial protocol, and predicts site enrollment, site default likelihood, and/or other related output for clinical trial sites based on the input data. In various embodiments, the MLM is any one of a regression model (e.g., linear regression, logistic regression, or polynomial regression), decision tree, random forest, gradient boosted machine learning model, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, the MLM is one of any one of a random forest model, an extremely randomized trees (XRT) model, a generalized linear model (GLM), a gradient boosting machine (GBM), XGBoost, a stacked ensemble, and a deep learning algorithm (e.g., fully connected multi-layer artificial neural network).

The MLM can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In particular embodiments, the machine learning implemented method is a logistic regression algorithm. In particular embodiments, the machine learning implemented method is a random forest algorithm. In particular embodiments, the machine learning implemented method is a gradient boosting algorithm, such as XGboost. In various embodiments, the model is trained using supervised learning algorithms, unsupervised learning algorithms, semi-supervised learning algorithms (e.g., partial supervision), weak supervision, transfer, multi-task learning, or any combination thereof.

In various embodiments, the MLM for analyzing selected features of the input data may include parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the machine learning models and the convolutional neural networks are trained (e.g., adjusted) using the training data to improve the predictive capacity of the machine learning model.

### V. Example Disease Indications and Clinical Trials

Embodiments disclosed herein are useful for identifying clinical trial sites that are likely to be high performing clinical trial sites. Thus, these high performing clinical trial sites can be enrolled in a clinical trial for investigating therapeutics for a variety of disease indications. In various embodiments, a disease indication for a clinical trial can include any one of immunology, cardiovascular and metabolic diseases, infectious diseases, oncology, and neuroscience. In particular embodiments, the disease indication is any one of Crohn's disease, lupus, diabetic kidney disease, lung cancer, or respiratory syncytial virus (RSV). Example clinical trials supported among the different therapeutic areas are: Tremfya for Crohn's Disease and Stelara for Lupus (Immunology), Invokana for DKD (CVM), JNJ 61186372/Lazertinib for Lung Cancer (Oncology) and VAC18193 for RSV (IDV).

### VI. Computer Implementation

The methods of the disclosed embodiments, including the methods of implementing MLM and a stochastic simulation for predicting clinical trial sites, are, in some embodiments, performed on one or more computers.

For example, the building and deployment of a MLM or a stochastic simulation can be implemented in hardware or software, or a combination of both. In one embodiment of the invention, a machine-readable storage medium is provided, the medium comprising a data storage material encoded with machine readable data which, when using a machine programmed with instructions for using said data, is capable of executing the training or deployment of machine learning models and/or displaying any of the datasets or results described herein. The embodiments can be implemented in computer programs executing on programmable computers, comprising a processor, and a data storage system (including volatile and non-volatile memory and/or storage elements). Some computing components (e.g., those used to display the user interfaces described herein may include additional components such as a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information. The databases of the present invention can be recorded on computer readable media, e.g., any medium that can be read and accessed directly by a computer. Such media include, but are not limited to magnetic storage media, hard disc storage medium, and magnetic tape; optical storage media; electrical storage media such as RAM and ROM; and hybrids of these categories such as magnetic/optical storage media. Any convenient data storage structure can be chosen, based on the means used to access the stored information. A variety of data processor programs and formats can be used for storage, e.g., word processing text file, database format, etc.

In some embodiments, the methods of the invention, including the methods for predicting enrollment of clinical trial sites, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 4 illustrates an example computer for implementing the entities shown in FIG. 1A, 1B, 2A, 2B, 3. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O) controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The network adapter 416 couples the computer 400 to one or more computer networks.

The graphics adapter 412 displays representation, graphs, tables, and other information on the display 418. In various embodiments, the display 418 is configured such that the user (e.g., data scientists, data owners, data partners) may input user selections on the display 418 to, for example, predict enrollment for a clinical trial site for a particular disease indication or order any additional exams or procedures. In one embodiment, the display 418 may include a touch interface. In various embodiments, the display 418 can show one or more predicted enrollments of a clinical trial site. Thus, a user who accesses the display 418 can inform the subject of the predicted enrollment of a clinical trial site.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIGS. 1A or 1B can vary depending upon the embodiment and the processing power required by the entity. For example, the site prediction system 130 can run in a single computer 400 or multiple computers 400 communicating with each other through a network such as in a server farm. The computers 400 can lack some of the components described above, such as graphics adapters 412, and displays 418.

### VII. Systems

Further disclosed herein are systems for implementing MLMs for generating site predictions for clinical trial sites. In various embodiments, such a system can include at least the site prediction system 130 described above in FIG. 1A. In various embodiments, the site prediction system 130 is embodied as a computer system, such as a computer system with example computer 400 described in FIG. 4 or other computer systems described herein.

### VIII. Example 1: Example Performance of Site Prediction Systems and Methods

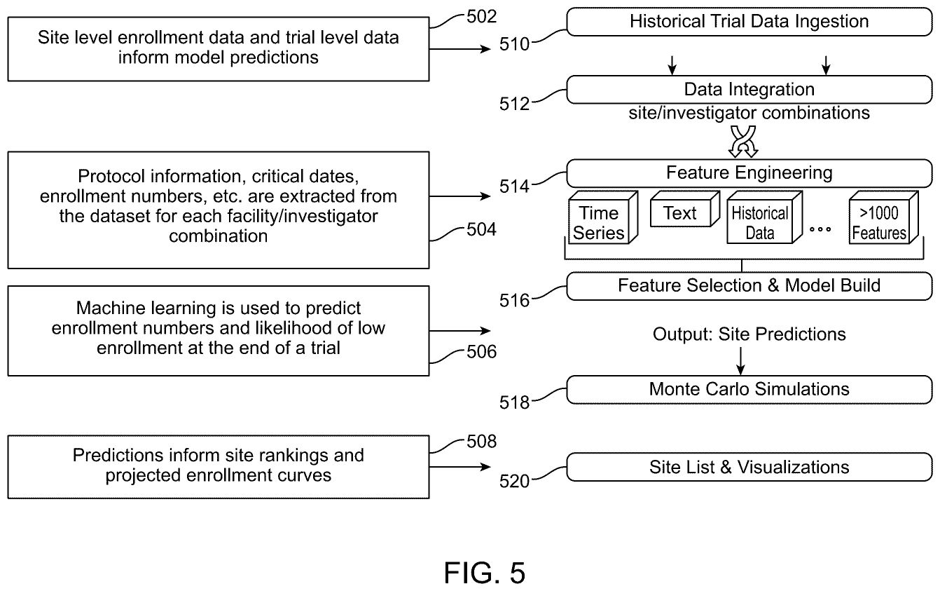

FIG. 5 depicts a first example of a site selection pipeline. As shown in FIG. 5, historical trial data such as site level enrollment data and trial level data were obtained 502 from resources such as DrugDev and Clinical Trials.gov, and ingested 510 by combining site or investigator combinations through data integration 512. The features of the ingested dataset, such as protocol information, critical dates, enrollment numbers, etc. were extracted 504 or selected from the dataset for each facility/investigator combination. For example, feature engineering 514 may be applied to select features 516 and build a model Machine learning was used to predict 506 enrollment numbers and likelihood of low enrollment at the end of a trial. The predictions from machine learning were informative of site rankings. Monte Carlo simulations 518 were used to predict 508 projected enrollment curves. The predictions of site rankings and projected enrollment curves were illustrated in a site list and visualized 520 in graphs and figures.

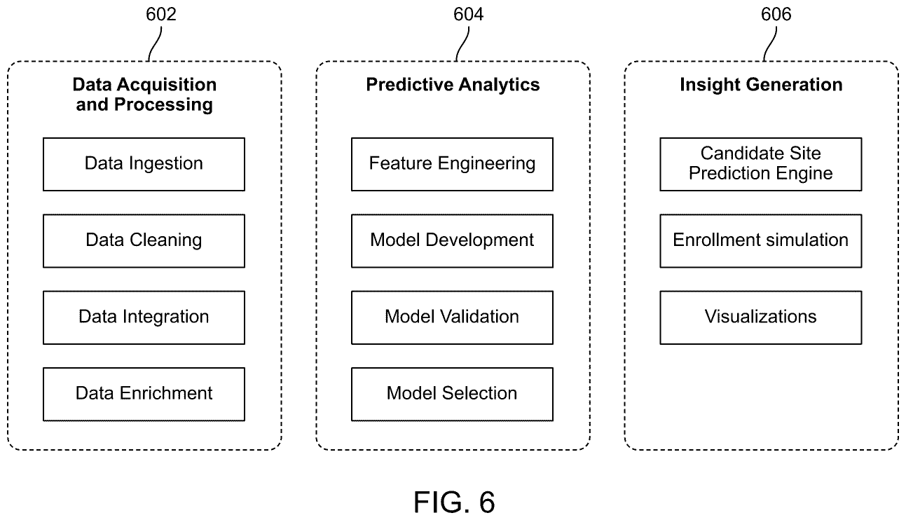

FIG. 6 depicts a site selection pipeline overview. As shown in FIG. 6, the site selection pipeline included data acquisition and processing 602 (e.g., data ingestion, data cleaning, data integration, and data enrichment), followed by predictive analytics 604 (e.g., feature engineering, model development, model validation, and model selection), and then insight generation 606 (candidate site prediction engine, enrollment simulation, and visualization). The data acquisition and processing steps 602 were performed by the data processing module 145 described in FIG. 1B. The predictive analytics 604 was performed by the feature engineering module 150 and model training module 155 according to methods described in reference to FIG. 1B. The insight generation steps 606 were performed by the model deployment module 160, the simulation module 165, and the visualization module 170 according to methods described in reference to FIG. 1B.

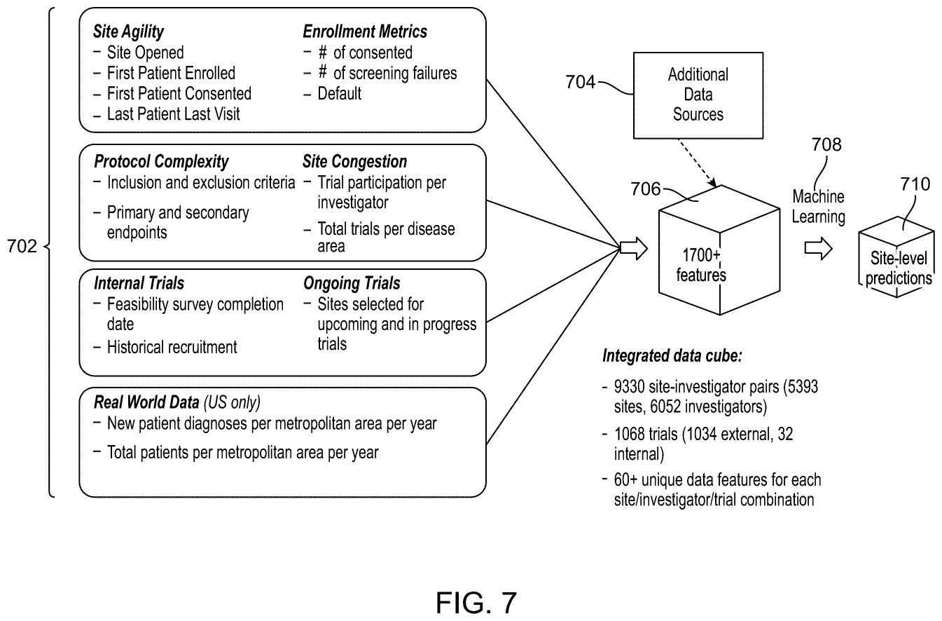

FIG. 7 depicts an example data ingestion process. As shown in FIG. 7, a large variety of clinical trial data informative of clinical trials and/or site-investigator pairs from multiple resources 702, 704 were ingested together in an integrated data cube 706. The integrated data cube included 9330 site-investigator pairs (5393 sites, 6052 investigators), 1068 trials (1034 external, 32 internal), and at least 60 unique data features for each site/investigator/trial combination. In some scenarios, the clinical trial data that were ingested can include additional or fewer resources. For example, the clinical trial data that were ingested need not include data from CTMS. A machine learning process 708 as described herein was applied to generate site-level predictions 710.

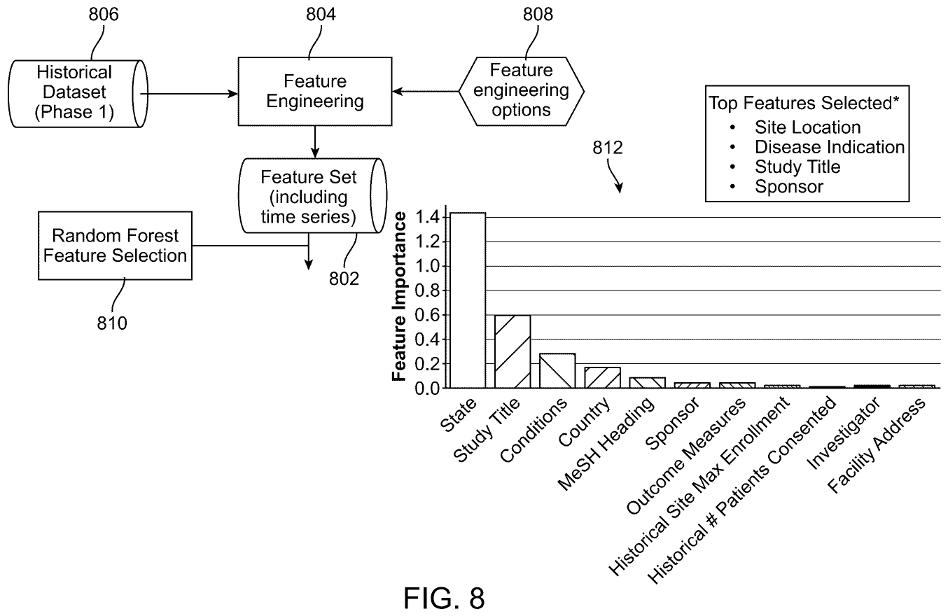

FIG. 8 depicts example selected features by feature engineering. A feature set including time series 802 was generated by applying feature engineering 804 to a historical dataset 806 and feature engineering options 808. A random forest feature selection 810 was then applied to the feature set to select features. As shown in the chart 812 of FIG. 8, example top features that were selected included site location, disease indication, study title, and sponsor.

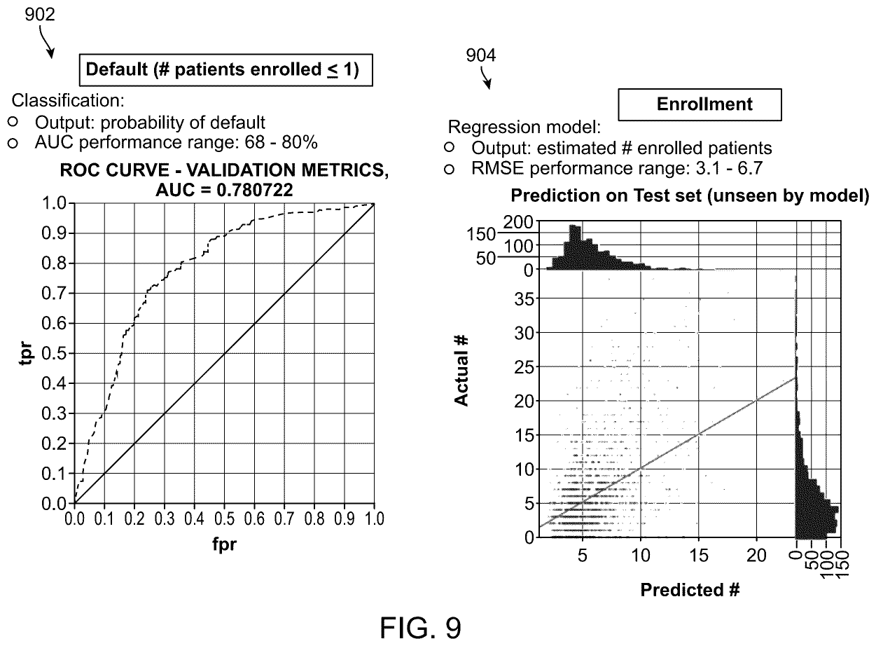

FIG. 9 depicts two example model performance for uses in model selection. As shown in FIG. 9, a model for predicting site default 902 (e.g., number of patients enrolled is zero or one), and a model for predicting site enrollment 904 were determined and chosen based on each model's performance (e.g., a best training score) where the model's interpretability was considered. As shown in FIG. 9, a classification model achieved an AUC performance metric of at least 0.68 for predicting likelihood of a site default or “probability of default.” A regression model achieved a root mean squared error (RMSE) performance metric between 3.1-6.7 for predicting estimated number of enrolled patients at a site or “estimated number of enrolled patients.”

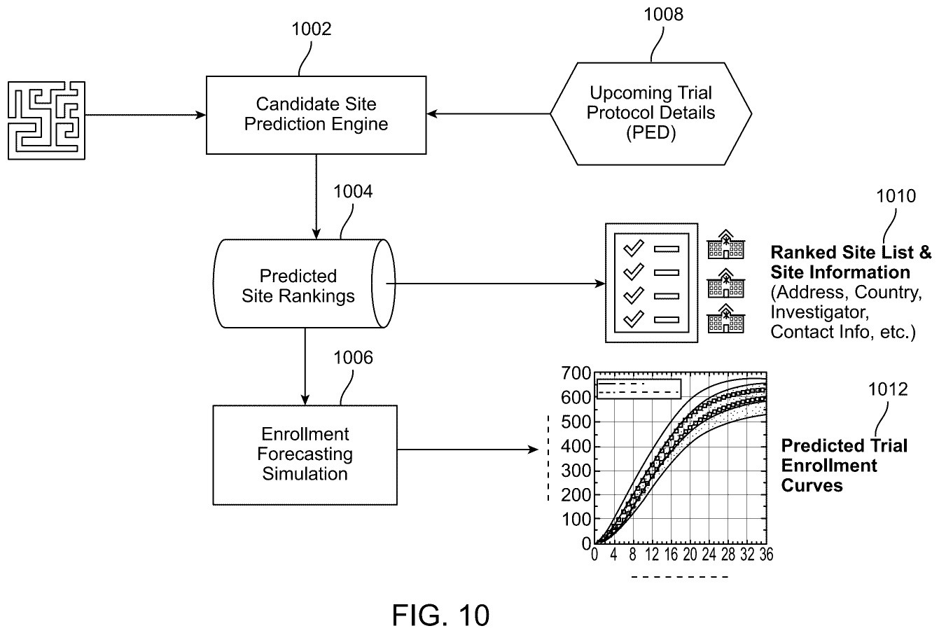

FIG. 10 depicts an example model deployment process. A candidate site prediction engine 1002 applied best performing machine learning models that were trained and selected from a plurality of candidate models to “upcoming trial protocol details (PED)” 1008 to generate predicted site rankings 1004. The predicted site rankings were illustrated in a ranked site list and site information (e.g., address, country, investigator, contact info, etc.) 1010. The predicted site rankings were then used as input to provide to stochastic simulations 1006 to generate enrollment forecasting simulation. The generated enrollment forecast was visualized as predicted trial enrollment curves on a graph 1012.

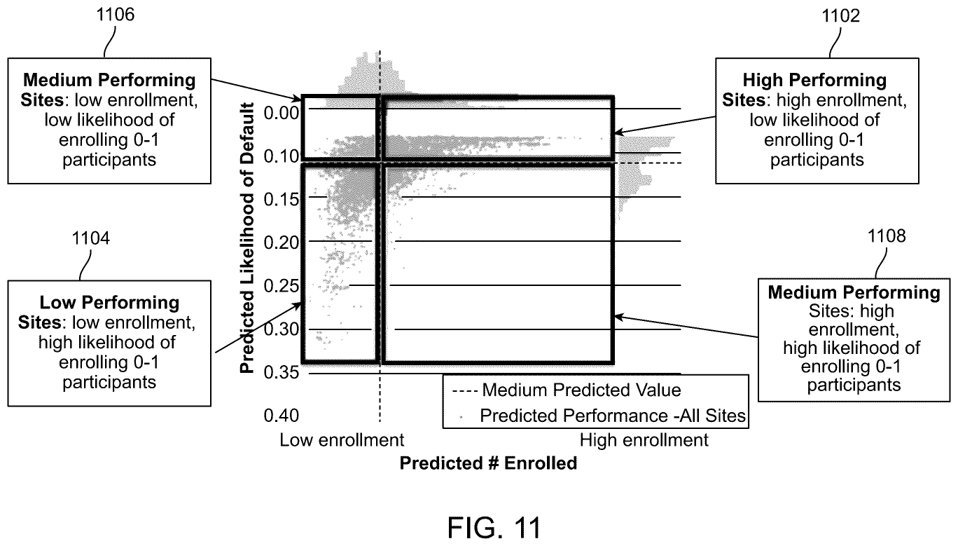

FIG. 11 depicts an example visualized site prediction for uses in site evaluation. As shown in FIG. 11, a quadrant graph was used to visualize two predicted variables: “predicted likelihood of default” and “predicted number of enrolled” for a plurality of clinical trial sites. In the quadrant graph, there were one “High Performing Sites” quadrant 1102, one “Low Performing Sites” quadrant 1104, and two “Medium Performing Sites” quadrants 1106, 1108. The clinical trial sites that fell in the “High Performing Sites” quadrant 1102 were predicted to have high enrollment and low likelihood of enrolling 0-1 participants, and the clinical trial sites that fell in the “Low Performing Sites” quadrant 1104 were predicted to have low enrollment, high likelihood of enrolling 0-1 participants. The clinical trial sites that fell in one “medium performing sites” quadrants 1106 were predicted to have low enrollment and low likelihood of enrolling 0-1 participants, and the clinical trial sites that fell in another “medium performing sites” quadrants 1108 were predicted to have high enrollment and high likelihood of enrolling 0-1 participants.

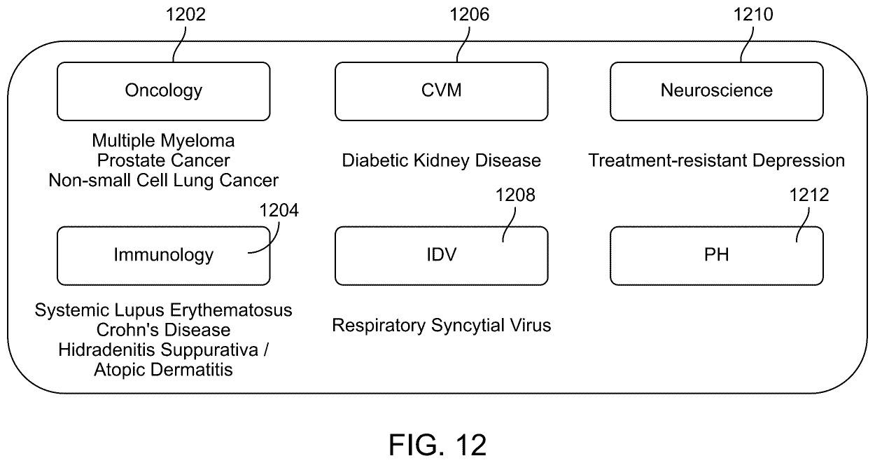

FIGS. 12-15 depict example clinical or therapeutic areas where the systems and methods in the presently disclosed embodiments were applied.

As shown in FIG. 12, the therapeutic areas included oncology 1202, immunology 1204, cardiovascular and metabolic diseases (CVM) 1206, infectious diseases (IDV) 1208, neuroscience 1210, PH 1212, etc. Example diseases in the therapeutic areas include multiple myeloma, prostate cancer, non-small cell lung cancer, diabetic kidney disease, treatment-resistant depression, systemic lupus erythematosus, Crohn's disease, hidradenitis suppurativa/atopic dermatitis, and respiratory syncytial virus.

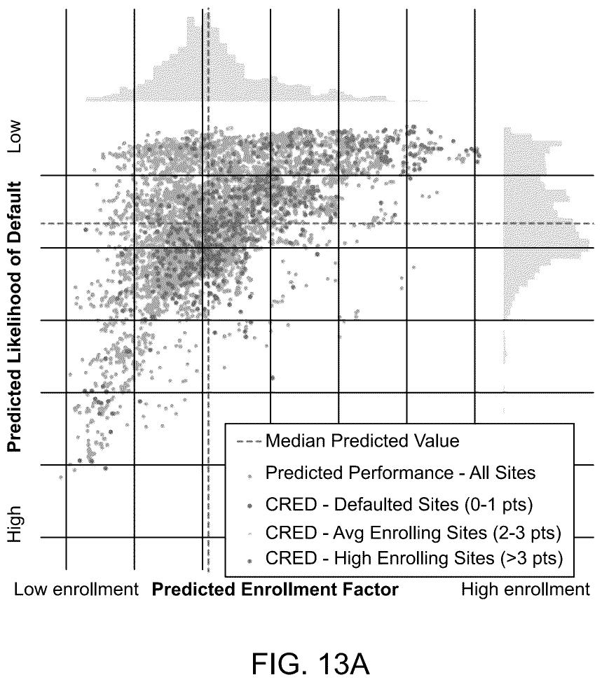

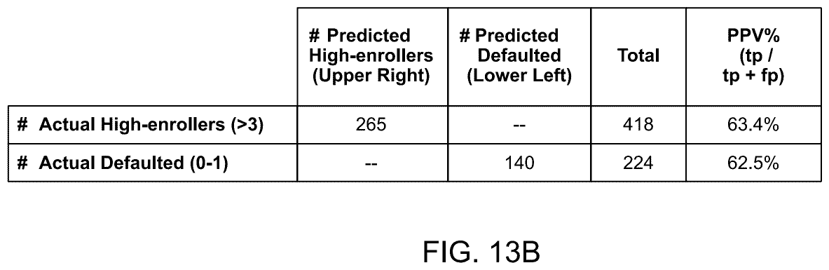

As shown in FIGS. 13A-B, the predictions from machine learning models were validated in a quadrant graph based on “Historical Trial” data. For “Recommended Sites (Upper Right Quadrant)” that fell in the high-performing sites quadrant, predictions from machine learning models achieved 63.4% positive predictive value (PPV) for true high-enrolling sites, resulting in an 15% improvement over site feasibility questionnaire estimates, and further achieved 17% false positive rate (FPR) for defaulted sites (vs 31% in the trial). For “Sites to Avoid (Lower Left Quadrant)” that fell in the low-performing sites quadrant, predictions from machine learning models achieved 62.5% PPV for true defaulted sites (140 sites). This demonstrated that the machine learning models described herein can be effectively deployed to identify a number of high-performing and low-performing sites.

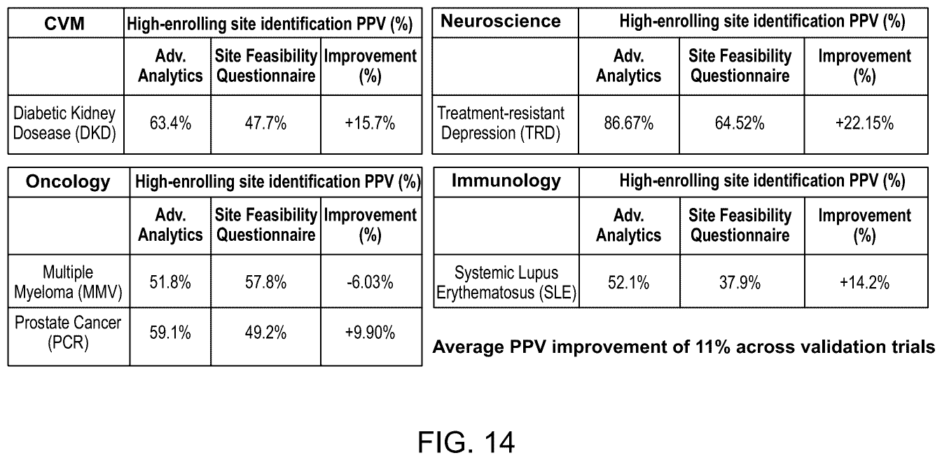

As shown in FIG. 14, predictions from machine learning models achieved improvement in identifying high-enrolling sites for clinical trials in a variety of clinical areas, such as 15.7% improvement for “diabetic kidney disease (DKD),” 22.15% improvement for “treatment-resistant depression (TRD)”, and 14.2% improvement for “systemic lupus erythematosus (SLE).” Predictions from machine learning models achieved an average PPV improvement of 11% across validation trials.

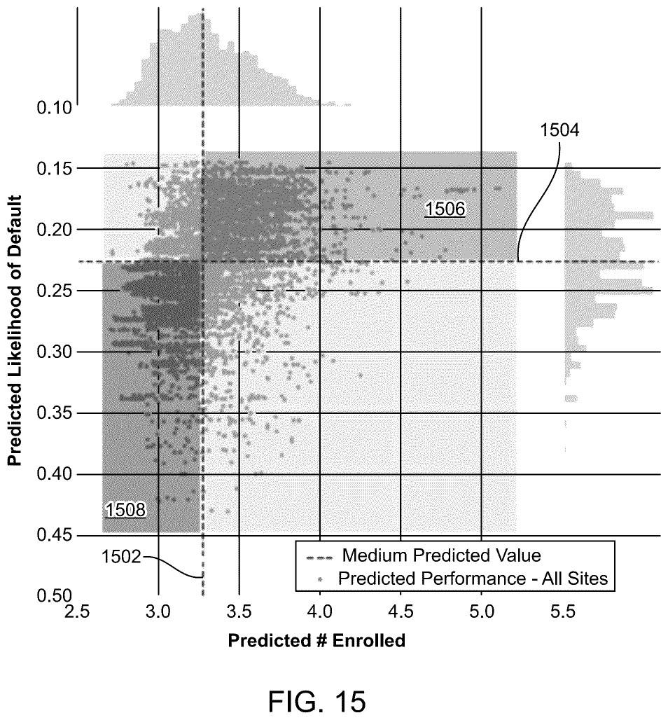

FIG. 15 depicts an example quadrant graph illustrating predictions from machine learning models. The predictions were generated for 6,368 sites and plotted on the quadrant graph including a dotted horizontal line 1504 representing median of predicted likelihood of default and a plotted vertical line 1502 representing median predicted number of patients enrolled. The quadrant graph showed that there were 2161 sites in the upper right quadrant 1506 and 2160 sites in the bottom left quadrant 1508, where sites in the upper right quadrant had higher chance of being high enrollers than the bottom left quadrant based on the predictions.

IX. Example 2: Example Site Prediction Systems and Methods for a particular disease Lupus

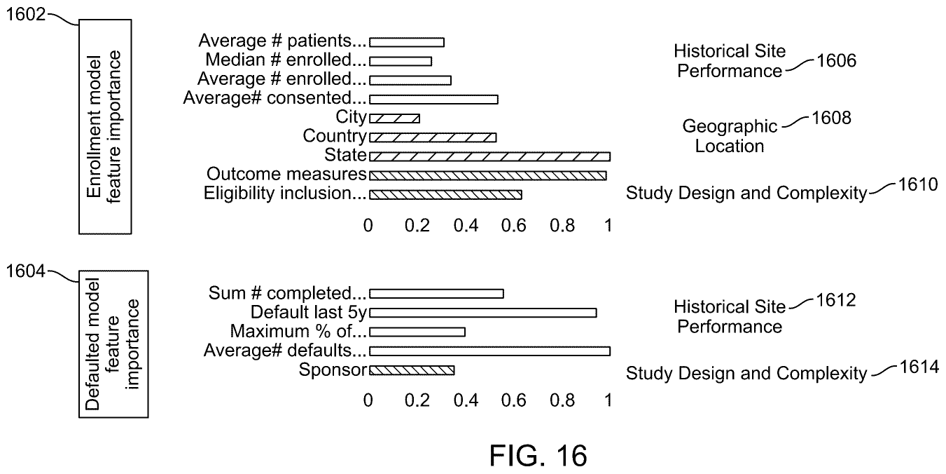

FIG. 16-19 depict example processes and results by applying systems and methods disclosed in the presently embodiments for a particular disease indication Lupus.

As shown in FIG. 16, multiple features were selected as input to provide to machine learning models to predict site enrollment 1602 and machine learning models to predict site default likelihood 1604. In some scenarios, selected features to provide to machine learning models to predict site enrollment included features informative of historical site performance 1606, geographic location (e.g., city, country, state) 1608, and study design and complexity (e.g., outcome measures, eligibility) 1610. In some scenarios, selected features to provide to machine learning models to predict site default likelihood included features informative of historical site performance 1612 and study design and complexity (e.g., sponsor) 1614.

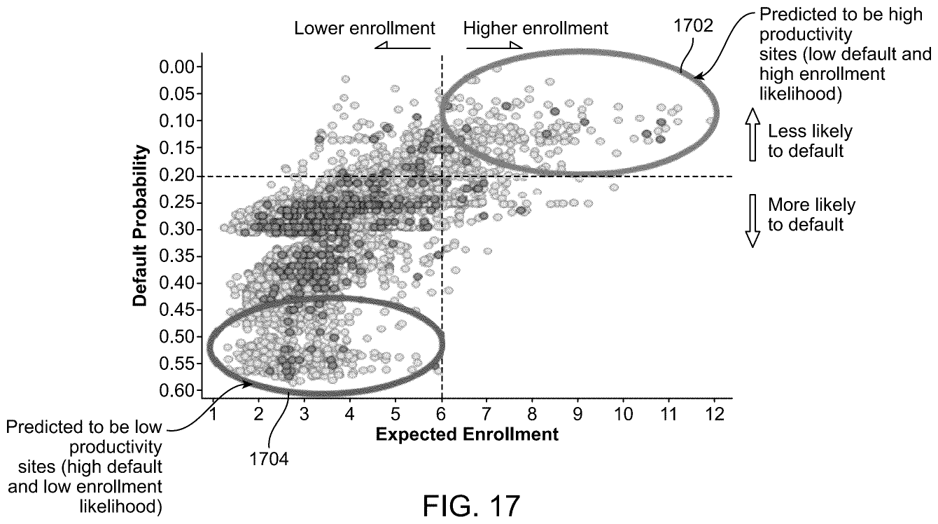

As shown in FIG. 17, a quadrant graph was used to visualize predictive site default and site enrollment for Lupus generated from machine learning models. The sites that were predicted to be high productivity sites 1702 (e.g., low default and high enrollment likelihood) fell in the right top quadrant. The sites that were predicted to be low productivity sites 1704 (e.g., high default and low enrollment likelihood) fell in the bottom left quadrant. Here, the thresholds used to establish the four quadrants were an expected enrollment of 6 and a default probability of 20%. The predictions were used as input to provide to a Monte Carlo simulation to predict enrollment timelines as discussed further in detail below.

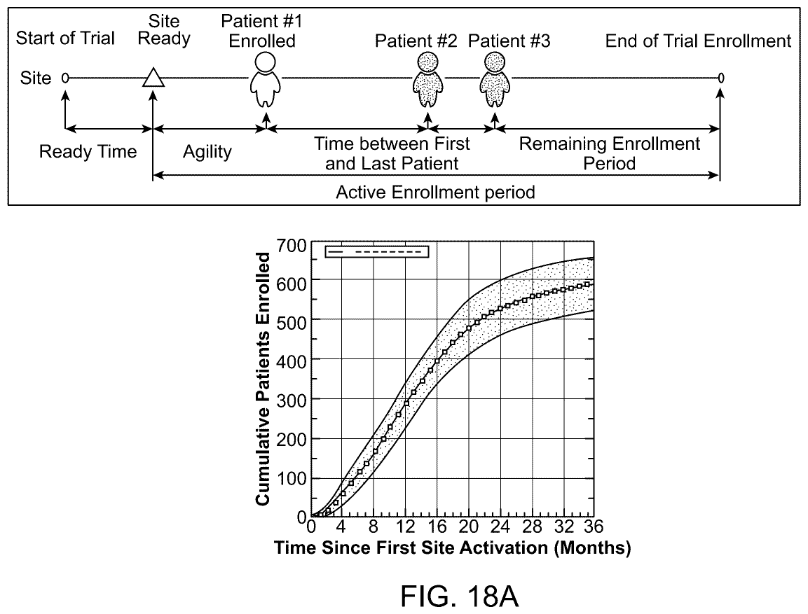

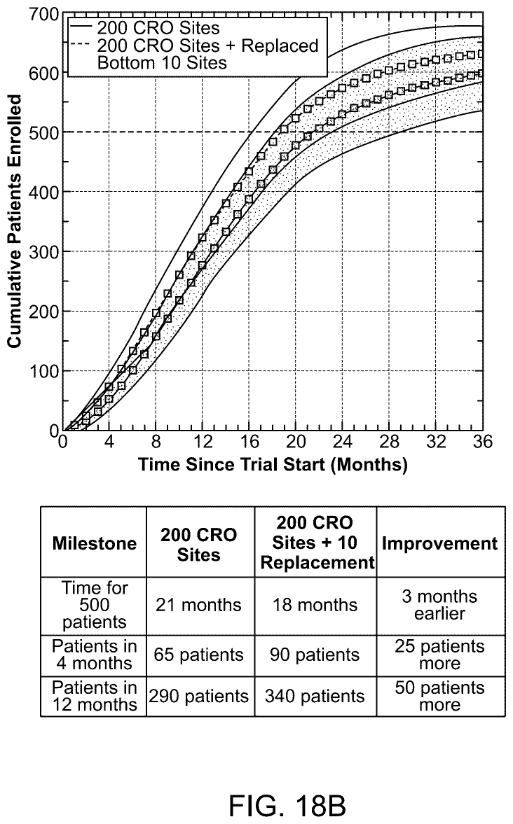

As shown in FIGS. 18A and 18B, the predictions from machine learning models were used as input to a Monte Carlo simulation to generate enrollment timelines. Insights were gathered by averaging over many independent simulation instances. In one scenario as shown in FIG. 18A, 500 patients and 200 sites worldwide were involved in the simulation. As shown in FIG. 18B, the simulation results predicted that if 10 of the 200 sites were replaced with high performing sites, the enrolment could be finished 3 months earlier. Additionally, at 4 months, the simulation results predicted 25 additional enrolled patients. Additionally, at 12 months, the simulation results predicted 50 additional enrolled patients.

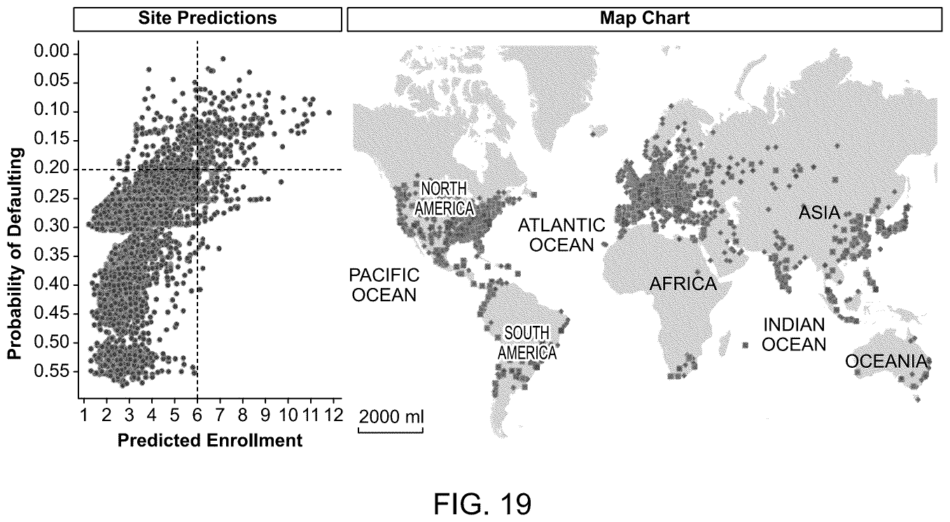

As shown in FIG. 19, the integrated dataset and predictions could be explored via Interactive Dashboards for further insights, such as evaluating sites that were previously selected.

The many different combinations of features and time windows that have been generated have resulted in improved performance of the models.

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

All references, issued patents and patent applications cited within the body of the instant specification are hereby incorporated by reference in their entirety, for all purposes.

1. An automated method for determining or selecting one or more clinical trial sites for inclusion in a clinical trial, comprising:
obtaining input data comprising data of an upcoming trial protocol;
for each of the one or more clinical trial sites:
generating a predicted site enrollment and a predicted site default likelihood for the clinical trial site by applying one or more machine learning models to selected features of the input data;
ranking the one or more clinical trial sites according to the predicted site enrollment and the predicted site default likelihood for the one or more clinical trial sites; and
selecting top-ranked clinical trial sites, wherein each of the selected clinical trial sites has a predicted site enrollment above a first threshold value and a predicted site default likelihood below a second threshold value,
wherein the selected features are previously determined by performing feature engineering on historical clinical trial data.

2. The method of claim 1, wherein the first threshold value is a median predicted site enrollment across the one or more clinical trial sites or a first specified value, and wherein the second threshold value is a median predicted site default likelihood across the one or more clinical trial sites or a second specified value.

3. The method of claim 1, further comprising:
generating a visualization of the predicted site enrollment and the predicted site default likelihood for the clinical trial sites in a quadrant graph.

4. The method of claim 1, further comprising:
generating a plurality of quantitative values informative of predicted enrollment timelines by applying a stochastic model to the predicted site enrollment and the predicted site default likelihood for the one or more clinical trial sites.

5. The method of claim 4, wherein the stochastic model comprises Monte Carlo simulation.

6. The method of claim 1, wherein the predicted site enrollment and the predicted site default likelihood are validated by using one or more of the historical clinical trial data and prospective clinical trial data.

7. The method of claim 1, further comprising:
generating a site list of the selected top-ranked clinical trial sites.

8. The method of claim 1, wherein the one or more machine learning models are determined by training a plurality of machine learning models, and by selecting the top performing model of the trained machine learning models.

9. The method of claim 1, wherein the one or more machine learning models are independently any one of a random forest model, an extremely randomized tree (XRT) model, a generalized linear model (GLM), a gradient boosting machine (GBM), XGBoost, a stacked ensemble, and a deep learning algorithm.

10. The method of claim 1, wherein the one or more machine learning models are trained to predict site enrollment and site default likelihood for a specific disease indication.

11. The method of claim 1, wherein the selected features comprise features associated with geographic locations, protocol complexity, study design, competitive landscape, and historical site enrollment metrics.

12. The method of claim 1, wherein the selected features comprise at least three of state of clinical trial site, study title, conditions, country, heading, sponsor, outcome measures, features associated with historical site enrollment metrics, investigator, and facility address.

13. The method of claim 12, wherein the features associated with historical site enrollment metrics comprise at least three of minimum, maximum, exponentially weighted moving average (EWMA), weighted average, and median of number of enrolled patients for a trial, number of patients consented for a trial, number of patients completed a trial, number of patients that failed screening for a trial, and agility over a reference time window at a reference entity.

14. The method of claim 1, wherein performing feature engineering on historical clinical trial data comprises:
converting trial metadata from the historical clinical trial data into a numerical representation of a single value or vector of values using n-grams, TFIDF, Word2vec, Glove, Fast Text, BERT, ELMo, InferSent.

15. The method of claim 1, wherein performing feature engineering on historical clinical trial data comprises:
applying a random forest feature selection algorithm to identify high importance features that have feature importance values above a threshold value.

16. The method of claim 1, wherein the predicted site enrollment comprises number of patients a site will enroll.

17. The method of claim 16, wherein the predicted site enrollment further comprises enrollment rate and/or agility, wherein the enrollment rate comprises number of patients per sit per month or year, and wherein the agility comprises time it took to start recruiting in a trial.

18. The method of claim 1, wherein the predicted site default likelihood comprises how likely a site is to enroll zero patients or fewer patients than a predetermined threshold.

19. A non-transitory computer-readable storage medium storing instructions for determining or selecting one or more clinical trial sites for inclusion in a clinical trial, the instructions when executed by a processor causing the processor to perform steps including:
obtaining input data comprising data of an upcoming trial protocol;
for each of the one or more clinical trial sites:
generating a predicted site enrollment and a predicted site default likelihood for the clinical trial site by applying one or more machine learning models to selected features of the input data;
ranking the one or more clinical trial sites according to the predicted site enrollment and the predicted site default likelihood for the one or more clinical trial sites; and
selecting top-ranked clinical trial sites, wherein each of the selected clinical trial sites has a predicted site enrollment above a first threshold value and a predicted site default likelihood below a second threshold value,
wherein the selected features are previously determined by performing feature engineering on historical clinical trial data.

20. A computer system comprising:
at least one processor, and
A non-transitory computer-readable storage medium storing instructions for determining or selecting one or more clinical trial sites for inclusion in a clinical trial, the instructions when executed by the at least one processor causing the at least one processor to perform steps including:
obtaining input data comprising data of an upcoming trial protocol;
for each of the one or more clinical trial sites:
generating a predicted site enrollment and a predicted site default likelihood for the clinical trial site by applying one or more machine learning models to selected features of the input data;
ranking the one or more clinical trial sites according to the predicted site enrollment and the predicted site default likelihood for the one or more clinical trial sites; and
selecting top-ranked clinical trial sites, wherein each of the selected clinical trial sites has a predicted site enrollment above a first threshold value and a predicted site default likelihood below a second threshold value,
wherein the selected features are previously determined by performing feature engineering on historical clinical trial data.
